(19) United States
(12) Patent Application Publication
(10) Pub. No.: US 2015/0017170 A1
(43) Pub. Date: Jan. 15, 2015

(54) BIOMARKER FOR SELECTING A SUBJECT FOR APPLICATION OF AN ANTI-C-MET ANTIBODY

(71) Applicant: Samsung Electronics Co., Ltd., Suwon-si (KR)

(72) Inventors: Young Mi OH, Seoul (KR); Kyung Ah Kim, Incheon (KR)

(21) Appl. No.: 14/327,172

(22) Filed: Jul. 9, 2014

(30) Foreign Application Priority Data
Jul. 9, 2013 (KR) 10-2013-0080281
Oct. 30, 2013 (KR) 10-2013-0130534

(51) Int. Cl. C12Q 1/37; A61K 45/06; A61K 39/395

(52) U.S. Cl. CPC C12Q 1/37; A61K 39/39558; A61K 45/06
USPC 424/138.1; 435/24

(57) ABSTRACT

A method of selecting a subject for administration of an anti-c-Met antibody, comprising (i) determining the presence or the amount of a ubiquitin peptidase, (ii) determining the presence or expression level of a ubiquitin peptidase coding gene, or (iii) measuring the activity of a ubiquitin peptidase, in a biological sample; as well as related methods.

### CROSS-REFERENCE TO RELATED APPLICATIONS

This application claims the benefits of Korean Patent Application No. 10-2013-0080281 filed on Jul. 9, 2013 in the Korean Intellectual Property Office, and Korean Patent Application No. 10-2013-0130534 filed on Oct. 30, 2013 in the Korean Intellectual Property Office, the entire disclosures of which are hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 162,967 bytes ASCII (Text) file named “715923_ST25.TXT,” created Jul. 8, 2014.

### BACKGROUND OF THE INVENTION

1. Field

A biomarker for selecting a subject for application of an anti-c-Met antibody, a method of selecting the subject for application of an anti-c-Met antibody including measuring a level of a ubiquitin peptidase and/or a ubiquitin peptidase coding gene, a method of preventing and/or treating a cancer including administering an anti-c-Met antibody to the selected subject, a pharmaceutical composition for preventing and/or treating a cancer including an anti-c-Met antibody and an inhibitor against a ubiquitin peptidase and/or a ubiquitin peptidase coding gene, and a method of preventing and/or treating a cancer including co-administering an anti-c-Met antibody and an inhibitor against a ubiquitin peptidase and/or a ubiquitin peptidase coding gene are provided.

2. Description of the Related Art

c-Met is a receptor for hepatocyte growth factor (HGF), a cytokine that binds the extracellular region of the c-Met receptor tyrosine kinase to induce cell division, movement, morphogenesis, and angiogenesis of various normal cells and tumor cells. c-Met is a representative receptor tyrosine kinase existing on the surface of cells, is itself a proto-oncogene, and is sometimes involved in various mechanisms related to cancer, such as cancer development, metastasis, migration, invasion, and angiogenesis, independent from the ligand, HGF.

In particular, c-Met is known to be involved in induction of resistance to commonly used anti-cancer drugs, and thus is regarded as important with respect to personalized treatments. Representative anti-cancer therapeutic drugs targeting epidermal growth factor receptor EGFR (ERBB1), i.e., Eribitux or Tarceva, work by blocking the signaling related to cancer development. In addition, Herceptin, which is well known as a breast cancer therapeutic drug, targets ERBB2 (HER2) and works by blocking the transduction of signals necessary for cell proliferation. Among patients resistant to the drugs described above, the signal transduction pathway that induces cell proliferation is not blocked due to the overexpression of c-Met. Thus, c-Met has emerged as a target of interest for many pharmaceutical companies, and antibodies against c-Met have been developed.

In order to increase the effect of therapies using the developed anti-c-Met antibodies, it is required to develop biomarkers for selecting a subject for application of the anti-c-Met antibody and/or for increasing the therapeutic effect of the anti-c-Met antibodies.

### BRIEF SUMMARY OF THE INVENTION

One embodiment provides a biomarker for selecting a subject for application of the anti-c-Met antibody (e.g., administration of or treatment with an anti-c-Met antibody), including a ubiquitin peptidase and/or a ubiquitin peptidase coding gene.

Another embodiment provides a composition and a kit for selecting a subject for application of the anti-c-Met antibody, including a means for detecting a ubiquitin peptidase and/or a ubiquitin peptidase coding gene and/or a means measuring the activity of a ubiquitin peptidase.

Another embodiment provides a method of selecting a subject for application of the anti-c-Met antibody (e.g., administration of or treatment with an anti-c-Met antibody), including determining the presence and/or the level of a ubiquitin peptidase (e.g., presence and/or amount of a ubiquitin peptidase) and/or a ubiquitin peptidase coding gene (e.g., presence and/or expression level of a ubiquitin peptidase coding gene), and/or measuring the activity of a ubiquitin peptidase.

Another embodiment provides a method of preventing and/or treating a cancer including administering an anti-c-Met antibody to the selected subject.

Another embodiment provides a pharmaceutical composition for preventing and/or treating a cancer including an anti-c-Met antibody and an inhibitor against a ubiquitin peptidase and/or a ubiquitin peptidase coding gene.

Another embodiment provides a method of preventing and/or treating a cancer including co-administering an anti-c-Met antibody and an inhibitor against a ubiquitin peptidase and/or a ubiquitin peptidase coding gene.

Another embodiment provides a method for screening a candidate drug for preventing and/or treating a cancer including determining the inhibition of a candidate compound against a ubiquitin peptidase and/or a ubiquitin peptidase coding gene.

Still another embodiment provides a method of enhancing the efficacy of an anti-c-Met antibody in preventing and/or treating cancer, including inhibiting a ubiquitin peptidase and/or a ubiquitin peptidase coding gene.

### BRIEF DESCRIPTION OF THE DRAWINGS

FIG. 1 is a photograph showing the results of immunoblotting (immunoprecipitation), which indicates the expression level of USP8 interacting with LRIG1 in LRIG1-overexpressed MKN45 cells after being treated with an anti-c-Met antibody.

FIG. 2 is a photograph showing the results of immunoblotting, which indicates the expression level of LRIG1 and USP8 in LRIG1- and USP8-overexpressed MKN45 cells after being treated with an anti-c-Met antibody.

FIG. 3 is a photograph showing the results of immunoblotting, which indicates the expression level of LRIG1, USP8, and ubiquitin in USP8-overexpressed EBC1 cells (left) and USP8 knock-down EBC1 cells (right) after being treated with an anti-c-Met antibody.

FIG. 4 is a graph showing cell viability (%) of USP8 knock-down EBC1 cells after being treated with an anti-c-Met antibody.

FIG. 5 is a photograph showing the results of immunoblotting, which indicates the expression level of LRIG1, USP8, and c-Met in wild type USP8-overexpressed EBC1 cells and mutated USP8-overexpressed EBC1 cells (right) after being treated with an anti-c-Met antibody.

FIG. 6 is a graph showing cell viability (%) of cells transfected with USP8-WT, USP8-CS or shUSP8 after being treated with an anti-c-Met antibody.

FIG. 7 is a graph showing the LRIG1 level in patient-derived lung tumor xenograft samples (#1, 2, 3, 4, 5, & 6).

FIG. 8 is a photograph showing the results of western blotting, which indicates the USP8 level in patient-derived lung tumor xenograft samples #1 and 2.

FIG. 9 is a graph showing the change of the c-Met level in patient-derived lung tumor xenograft samples #1 and 2 by treating with an anti-c-Met antibody.

FIG. 10 is a graph showing the change of the tumor volume in patient-derived lung tumor xenograft samples #1 and 2 by treating with an anti-c-Met antibody (Asterisks (*): P-values versus vehicle group according to repeated measures ANOVA (*P<0.05, **P<0.01, ***P<0.001, ****P<0.0001)).

### DETAILED DESCRIPTION OF THE INVENTION

Anti-c-Met antibodies can induce c-Met degradation in a LRIG1-dependent manner. When anti-c-Met antibodies induce c-Met degradation through LRIG1, c-Met activation is not required. Thus, re-use of c-Met can be prevented and the anticancer efficacy of the anti-c-Met antibody can be maximized.

Without wishing to be bound by any particular theory or mechanism of action, it is believed that anti-c-Met antibodies, particularly those described herein, can effectively lower the c-Met level in a subject in a LRIG1-dependent manner, wherein ubiquitination of LRIG1 is induced. It is further believed that anti-c-Met antibodies inhibit the interaction between LRIG1 and ubiquitin peptidase (e.g., ubiquitin specific peptidase (USP) family), whereby the ubiquitination of LRIG1 is induced.

Therefore, in anticancer therapy using an anti-c-Met antibody, specifically an anti-c-Met antibody acting in a LRIG1-dependent manner, when a patient group having high levels of LRIG1 and low levels of ubiquitin peptidase, relative to a reference sample as described below, is selected and treated with the specific antibody, more successful therapy by LRIG1-dependent c-Met targeting mechanism of the antibody can be achieved. In this case, the decrease of interaction between ubiquitin peptidase and LRIG1 leads to stimulation ubiquitination of LRIG1, thereby contributing to c-Met degradation by LRIG1.

Therefore, the presence/absence and/or level of ubiquitin peptidase can be utilized as a molecular marker (biomarker) in therapy using anti-c-Met antibodies.

One embodiment provides a use of a ubiquitin peptidase and/or a gene for a ubiquitin peptidase as a biomarker for selecting (identifying) a subject for application of an anti-c-Met antibody.

In particular, a biomarker composition for selecting (identifying) a subject for application of an anti-c-Met antibody, including a ubiquitin peptidase and/or a gene for a ubiquitin peptidase is provided.

Another embodiment provides a kit for selecting (identifying) a subject for application of an anti-c-Met antibody, including a means for detecting a ubiquitin peptidase and/or a gene for a ubiquitin peptidase and/or a means measuring the activity of a ubiquitin peptidase.

Another embodiment provides a method of selecting (identifying) a subject for application of an anti-c-Met antibody including determining the presence and/or the level (or amount) of a ubiquitin peptidase and/or a ubiquitin peptidase coding gene and/or measuring the activity of a ubiquitin peptidase, in a biological sample.

In the selection of a subject for application of an anti-c-Met antibody, when a ubiquitin peptidase (e.g., USP8) and/or a gene for a ubiquitin peptidase is absent or present at a low level in the biological sample, the biological sample or the patient from which the biological sample is obtained (separated) may be considered (or selected as) a suitable subject for application of an anti-c-Met antibody. For example, the level of a ubiquitin peptidase (e.g., USP8) may be determined by immunohistochemical staining using a general antibody against the ubiquitin peptidase. The immunohistochemical staining is a method for identifying a material present in a cell or a tissue using antigen-antibody response, wherein a frozen or paraffin tissue section is generally used. A tissue section having a regular thickness is blocked for preventing non-specific binding of an antibody, and then, treated with a primary antibody. After a certain period, non-reacting primary antibody is removed, and the tissue section is treated with a secondary antibody. The secondary antibody attached tissue section can be detected using a streptavidin-attached material, such as streptavidin-HRP or streptavidin-alkaline phosphatase, which can bind to biotin attached to the secondary antibody. Most of the detecting responses are color reactions, which can be analyzed by a proper microscope. The staining may be scored on a scale ranging, e.g., from ‘−’ or ‘0’ to ‘+3,’ wherein a score (stain intensity) of ‘−’ or ‘0’ represents no protein expression (no signal, negative), score of ‘+1’ represents no or a slight protein expression (corresponding to a background signal), and scores of ‘+2’ (strongly positive) to ‘+3’ (very strongly positive) represent progressively increased levels of protein expression (the case showing the signal higher than ‘+3’ is incorporated in the score of ‘+3’) (the scores can be determined by a pathologist). Thus, when the score measured by immunohistochemical staining is “−”, “0”, or “+1”, the level of the ubiquitin peptidase may be determined as “negative”, and when the score measured by immunohistochemical staining is greater than “+1” (i.e., “+2”, or “+3”), the level of the ubiquitin peptidase may be determined as “positive”, where the “negative” may be understood as absence, or presence at a low level, of the ubiquitin peptidase (e.g., USP8) in the biological sample. Therefore, the above “absence, or presence at a low level, in the biological sample” may correspond to the case that the score measured by immunohistochemical staining using an antibody against the ubiquitin peptidase is “−”, “0”, or “+1”.

Alternatively, the “low level of ubiquitin peptidase (e.g., USP8) and/or a gene for ubiquitin peptidase or an activity of ubiquitin peptidase” may be determined by comparing the level of ubiquitin peptidase (e.g., USP8) in a biological sample from a subject with that in a reference sample. The reference sample may be any in which a c-Met inhibitor such as an anti-c-Met antibody has no effect or having a resistance to a c-Met inhibitor. For example, the reference sample may be at least one selected from the group consisting of cell lines H1373 (ATCC, CRL-5866), Caki-1 (ATCC, HTB-46), BT474 (ATCC, HTB-20), HT-29 (ATCC, HTB-38), SW620 (ATCC, CCL-227), Ls174T (ATCC, CL-188), and c-Met inhibitor resistant cells (e.g., cells acquiring a resistance to a c-Met inhibitor by repeated and/or consistent administration of the c-Met inhibitor). Therefore, the method of selecting (identifying) a subject for application of an anti-c-Met antibody may further include a step of comparing the level of ubiquitin peptidase and/or an ubiquitin peptidase coding gene in a biological sample from a patient with that of a reference sample as described above. In this case, the method may further include a step of measuring the level of ubiquitin peptidase and/or an ubiquitin peptidase coding gene or an activity of ubiquitin peptidase, in the reference sample. The method may further include a step of determining (considering or selecting) the biological sample or the patient from which the biological sample is obtained (separated) as a suitable subject for application of an anti-c-Met antibody, when ubiquitin peptidase and/or an ubiquitin peptidase coding gene or an activity of ubiquitin peptidase is absent in the biological sample, or present at a low level in the biological sample compared to that of the reference sample.

The enzymatic activity of the ubiquitin peptidase (e.g., USP8) relating to ubiquitination (binding of ubiquitin) of a target is important. Therefore, the subject, whose a ubiquitin peptidase has no enzymatic activity or low enzymatic activity due to a mutation in an active site of the enzyme, and the like—thereby leading to no effect of ubiquitination of a target or low level of the ubiquitination)—may be determined to be suitable for the application of the anti-c-Met antibody. Therefore, the step of measuring the activity of a ubiquitin peptidase may include a step of determining the presence or absence of a mutation in an active site of a ubiquitin peptidase (e.g., USP8), or measuring the degree of ubiquitination of a target (e.g., LRIG1). For example, the mutation in an active site of a ubiquitin peptidase (e.g., USP8) may be a substitution of the amino acid residue Cys at the 786^{th} position of NP_{—}001122082 (human USP8; SEQ ID NO: 109) with Ser (C786S). When the mutation or a mutant having the mutation is found in the biological sample, or the ubiquitination of a target (e.g., LRIG1) is found at a low level or not at all in the biological sample, the biological sample or a patient from which the biological sample is obtained (separated) may be considered a suitable subject for the application of an anti-c-Met antibody.

The subject for the application of an anti-c-Met antibody may be any subject suitable for the application of a therapy using an anti-c-Met antibody, and for example, selected from the group consisting of any mammals including primates such as human, and monkey; and rodents such as mice and rats. In one specific embodiment, the subject may be a cancer patient. The biological sample may be the subject, or a cell, a tissue, or body fluid (e.g., blood, serum, saliva, urinary, etc.) derived (separated) from the subject. Specifically, biological sample may be a cancer cell or a cancer tissue.

The ubiquitin peptidase may be any enzyme capable of inducing the degradation of ubiquitin in vivo or ex vivo. It may be USP (Ubiquitin specific peptidase), and specifically, USP8 (Ubiquitin specific peptidase 8). USP8 is a typical deubiquitinating enzyme, and associated with a degradation mechanism of a target by deubiquitination of various cell membrane proteins.

A gene for a ubiquitin peptidase, which encodes a ubiquitin peptidase, for example, USP (Ubiquitin specific peptidase), specifically USP8, may be at least one selected from the group consisting of full-length DNA, cDNA, and mRNA.

It is believed that inhibition of the USP8 gene (for example, by knock-down) may induce a decreased interaction between USP8 and LRIG1, thereby promoting the ubiquitination of LRIG1, when an anti-c-Met antibody is applied as part of treatment, c-Met degradation accelerates, which contributes to the increased anticancer effect of the anti-c-Met antibody. Therefore, in one embodiment USP8 and/or a gene encoding USP8 may be used as a biomarker for selecting a subject for application of an anti-c-Met antibody.

USP8 may be derived from mammals such as primates, including humans and monkeys, and also rodents, including mice and rats. For example, USP8 may be a human USP8 comprising the amino acid sequence of NCBI Accession No. NP_{—}001122082 (SEQ ID NO: 109) or the amino acid sequence encoded by the nucleotide sequence (mRNA) of NM_{—}001128610 (SEQ ID NO: 110; coding domain: from 339^{th} to 3695^{th} positions), a mouse USP8 comprising the amino acid sequence of NP_{—}001239509 or the amino acid sequence encoded by the nucleotide sequence (mRNA) of NM_{—}001252580 or BC066126.

The measurement of the absence/presence and the level of a ubiquitin peptidase and/or a gene for a ubiquitin peptidase, and the activity of a ubiquitin peptidase may be performed by measuring using any ordinary means for a gene or protein quantitative assay or for measuring an enzymatic activity, and/or by evaluating the measured results. For example, the absence/presence and the level of a ubiquitin peptidase (e.g., USP8) may be measured via an ordinary enzyme reaction, fluorescence, luminescence, and/or radioactivity detection using at least one selected from the group consisting of ubiquitin peptidase specific antibodies, and aptamers. More particularly, it may be measured by a method selected from the group consisting of immunochromatography, immunohistochemistry, enzyme linked immunosorbent assay (ELISA), radioimmunoassay (RIA), enzyme immunoassay (EIA), fluorescence immunoassay (FIA), luminescence immunoassay (LIA), and western blotting, but is not limited thereto. In addition, the absence/presence and the level of a gene for a ubiquitin peptidase may be measured by using any ordinary gene quantification methods including, but not limited to, an ordinary polymerase chain reaction (PCR), FISH (fluorescent in situ hybridization) using a primer, probe or aptamer, which is hybridizable with the gene.

In a particular embodiment, the primer may be able to detect a gene fragment of 5 to 1000 bp, 10 to 500 bp, 20 to 200 bp, or 50 to 200 bp within the nucleotide sequence of a gene coding for a ubiquitin peptidase, for example USP8 gene (full-length DNA, cDNA, or mRNA), and may comprise or consist essentially of a nucleotide sequence hybridizable with (complementary to) a region of 5 to 100 bp, 5 to 50 bp, 5 to 30 bp, or 10 to 25 bp of the 3′-end and/or 5′-end of the gene fragment. The probe or aptamer capable of hybridizing with the gene may comprise or consist essentially of a nucleotide sequence with a size of from about 5 to about 100 bp, from about 5 to about 50 bp, from about 5 to about 30 bp, or from about 5 to about 25 bp, which is capable of hybridizing with (or is complementary to) a fragment (about 5 to about 100 bp, about 5 to about 50 bp, about 5 to about 30 bp, or about 5 to about 25 bp) of the USP8 gene (full-length DNA, cDNA or mRNA). As used herein, the term “capable of hybridizing” may refer to complementarily binding to a specific region of the gene, with a sequence complementarity of 80% or higher, e.g., 90% or higher, 95% or higher, 98% or higher, 99% or higher, or 100% between the primer, probe or aptamer and the gene region.

The means for detecting a ubiquitin peptidase and/or a ubiquitin peptidase coding gene and/or a means measuring the activity of a ubiquitin peptidase may be any ordinary means used in the above described methods for measuring the absence/presence or the level of a ubiquitin peptidase and/or a gene for a ubiquitin peptidase or the activity of a ubiquitin peptidase.

In a particular embodiment, the activity of a ubiquitin peptidase may be measured by determining the presence or absence of a mutation of a ubiquitin peptidase (e.g., USP8) in an active site affecting its activity, or the level of ubiquitination of a target. The activity may be a mutation where amino acid residue Cys at 786^{th} position of NP_{—}001122082 (human USP8; SEQ ID NO: 109) is substituted with Ser (C786S). For example, the enzymatic activity of a ubiquitin peptidase may be determined by measuring the ubiquitination of a target, i.e., the presence or the level of the target (e.g., LRIG1) to which ubiquitin binds. The measurement of the presence or the level of the target (e.g., LRIG1) to which ubiquitin binds may be performed via an ordinary enzyme reaction, fluorescence, luminescence, and/or radioactivity detection using at least one selected from the group consisting of LRIG1 specific antibodies, and aptamers. Certain specific methods are as described above. In addition, the enzymatic activity of a ubiquitin peptidase may be determined by an ordinary means capable of confirming the presence of the mutation described above and/or a mutant including the mutation. Accordingly, it is possible to select a patient group having a high level of LRIG1 and a low level of USP8, determined by measuring the amounts of LRIG1 and USP from a protein sample (about 5 ug) or RNA sample (about 1 ug) extracted from only a small amount of tissue or cells. This can increase the clinical efficiency of clinical c-Met targeting anticancer therapy. The method of selecting a subject for application of the anti-c-Met antibody may be extended to HGF/c-Met inhibitors other than the anti-c-Met antibody as well.

Another embodiment provides a method of c-Met inhibition or a method of preventing and/or treating a cancer comprising administering an anti-c-Met antibody to the selected subject.

The method of c-Met inhibition or the method of preventing and/or treating a cancer may further comprise a step of identifying a subject for application of the anti-c-Met antibody prior to the step of administration. The step of identification may be performed by the method of selecting a subject for application of the anti-c-Met antibody as described above.

In an embodiment, the method of c-Met inhibition or the method of preventing and/or treating a cancer may include:

identifying a subject for the application of an anti-c-Met antibody; and

administering an effective amount of an anti-c-Met antibody to the subject.

In another embodiment, the method of c-Met inhibition or the method of preventing and/or treating a cancer may include:

selecting a subject for the application of an anti-c-Met antibody by measuring the absence/presence and the level of a ubiquitin peptidase (e.g., USP8) and/or a gene for a ubiquitin peptidase, and the activity of a ubiquitin peptidase (e.g., USP8); and

administering an effective amount of an anti-c-Met antibody to the selected subject.

In a therapy using an anti-c-Met antibody, when ubiquitin peptidase (e.g., USP8) and/or a gene for a ubiquitin peptidase is absent or present at a low level, or the activity of a ubiquitin peptidase (e.g., USP8) is low or lost, more excellent therapeutic effect can be achieved. Therefore, the co-administration of an anti-c-Met antibody with an inhibitor against a ubiquitin peptidase (e.g., USP8) and/or a gene for a ubiquitin peptidase may lead to a synergistic effect.

Another embodiment provides a pharmaceutical composition for combination administration for preventing and/or treating a cancer comprising an anti-c-Met antibody and an inhibitor against a ubiquitin peptidase and/or a ubiquitin peptidase coding gene as active ingredients.

In one embodiment, the pharmaceutical composition for combination administration may be in a form for simultaneous administration of two drugs including a mixture of a pharmaceutically effective amount of an anti-c-Met antibody and a pharmaceutically effective amount of an inhibitor against a ubiquitin peptidase and/or a ubiquitin peptidase coding gene.

In another embodiment, the pharmaceutical composition for combination administration may be in a form of simultaneous or sequential administration of a pharmaceutically effective amount of an anti-c-Met antibody and a pharmaceutically effective amount of an inhibitor against a ubiquitin peptidase and/or a ubiquitin peptidase coding gene, each being individually formulated. In this case, the pharmaceutical composition for combination administration may be a pharmaceutical composition for combination administration for simultaneous or sequential administration including a first pharmaceutical composition containing a pharmaceutically effective amount of an anti-c-Met antibody and a second pharmaceutical composition containing a pharmaceutically effective amount of an inhibitor against a ubiquitin peptidase and/or a ubiquitin peptidase coding gene. In the case of sequential administration, it can be performed in any order.

Another embodiment provides a kit for preventing and/or treating cancer, including a first pharmaceutical composition containing a pharmaceutically effective amount of an anti-c-Met antibody, a second pharmaceutical composition containing a pharmaceutically effective amount of an inhibitor against a ubiquitin peptidase and/or a ubiquitin peptidase coding gene, and a package container.

Another embodiment provides a method for preventing and/or treating cancer comprising co-administering a pharmaceutically effective amount of an anti-c-Met antibody or an antigen-bonding fragment thereof and a pharmaceutically effective amount of an inhibitor against a ubiquitin peptidase and/or a ubiquitin peptidase coding gene to a subject in need of prevention and/or treatment of cancer. The method may further include a step of identifying a subject who is in need of the prevention and/or treatment of cancer, prior to the administration step.

The combination administration step may be performed either by administering an anti-c-Met antibody and an inhibitor against a ubiquitin peptidase and/or a ubiquitin peptidase coding gene together (at the same time) or by administering them sequentially in any order. In one embodiment, the combination administration may be performed by administering a mixture of a pharmaceutically effective amount of an anti-c-Met antibody and a pharmaceutically effective amount of an inhibitor against a ubiquitin peptidase and/or a ubiquitin peptidase coding gene. In another embodiment, the combination administration may be done by performing a first step of administering a pharmaceutically effective amount of an anti-c-Met antibody and a second step of administering a pharmaceutically effective amount of an inhibitor against a ubiquitin peptidase and/or a ubiquitin peptidase coding gene simultaneously or sequentially. In the case of sequential administration, it can be performed in any order.

The subject may be mammals such as primates, including humans and monkeys, and rodents, including mice and rats, or cells or tissues isolated from the living body thereof.

Another embodiment provides for the use of combination administration of an anti-c-Met antibody and an inhibitor against a ubiquitin peptidase and/or a ubiquitin peptidase coding gene in preventing and/or treating cancer.

In accordance with the invention, by co-administering an anti-c-Met antibody and an inhibitor against a ubiquitin peptidase and/or a ubiquitin peptidase coding gene, excellent synergistic effects can be achieved in comparison with the use of the anti-c-Met antibody alone. Furthermore, even when administration concentrations are decreased and/or administration intervals are extended, at least equivalent effects can be obtained in comparison with the use of a single drug, and side effects against the anti-c-Met antibody can be minimized.

The inhibitors against a ubiquitin peptidase and/or a ubiquitin peptidase coding gene may be any compound(s) capable of inhibiting the expression and/or activity of the ubiquitin peptidase and/or the gene. For example, in the case of the gene, the inhibitors may be one or more selected from the group consisting of a chemical inhibitor (compound drug) against the gene, an siRNA against the gene, a microRNA against the gene, an shRNA against the gene, and an aptamer against the gene. For example, they may be one or more selected from the group consisting of an siRNA, microRNA, shRNA, and aptamer, all of which are capable of hybridizing with adjacent 2 to 200 bp, particularly 10 to 100 bp or 20 to 50 bp regions within the nucleotide sequences of the above genes. The ‘capable of hybridizing’ or ‘hybridizable’ refers to when complementary binding is possible by having sequence homology of at least 80%, at least 90%, at least 95%, at least 98%, at least 99%, or 100% to the nucleotide sequences of the above gene regions. Also, in the case of the ubiquitin peptidase, the inhibitors may be one or more selected from the group consisting of a mutant of the ubiquitin peptidase having a mutation in an active site (for example, a mutant containing a substitution of at least one amino acid within the active site of the ubiquitin peptidase with different amino acid), a chemical inhibitor (e.g., synthetic compound drug) against the ubiquitin peptidase, an antibody against the ubiquitin peptidase, or an aptamer against the gene or the ubiquitin peptidase.

For example, the inhibitor may be at least one selected from the group consisting of shRNAs specifically binding to a gene encoding a ubiquitin peptidase, particularly USP8, for example, shRNA including the nucleotide sequence of 5′-tatctcttccgattatcag-3′ (SEQ ID NO: 112); shUSP8 mature antisense). In another example, the inhibitor may be at least one selected from the group consisting of USP8-specific inhibitors such as HBX 90,397 or HBX 90,659 (see WO2007017758), a General de-ubiquitinating enzyme (DUB) inhibitor such as PR-619, but not limited thereto.

HBX 90,397 has the following structure:

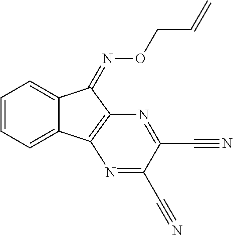 [SMILES: C=CCO/N=C1/c2ccccc2-c2nc(C#N)c(C#N)nc21]

PR-619 has the following structure:

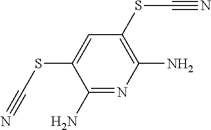 [SMILES: N#CSc1cc(SC#N)c(N)nc1N]

In another example, the inhibitor may be a mutant containing a mutation in an active site or a polynucleotide encoding the mutant. For example, the mutant may be a USP8 mutant (SEQ ID NO: 111) where amino acid residue Cys at 786^{th} position of USP8, for example NP_{—}001122082 (human USP8; SEQ ID NO: 109) is substituted with Ser (C786S), or a polynucleotide encoding the mutant. The polynucleotide encoding the mutant may be inserted into a proper vector, and used for transfection (in vivo or ex vivo) by replacing a normal USP8 gene of a separated cell.

As described above, the selected subject for the application of an anti-c-Met antibody may be one having no or a low level of a ubiquitin peptidase and/or a gene for a ubiquitin peptidase or having no or a low level of ubiquitin peptidase activity a, wherein the no or low level of a ubiquitin peptidase and/or a gene for a ubiquitin peptidase or no or low enzymatic activity level may be inherent or achieved by artificial treatments for removing or lowering the ubiquitin peptidase and/or the gene or the enzymatic activity (e.g., mutation of USP8 as described above). Therefore, an artificial inhibition or mutation of a ubiquitin peptidase and/or a gene for a ubiquitin peptidase may make the subject suitable for the application of an anti-c-Met antibody or sensitive to the therapy using the anti-c-Met antibody.

Therefore, another embodiment provides a composition for enhancing (increasing) an efficacy of an anti-c-Met antibody, including an inhibitor against a ubiquitin peptidase (e.g., USP8) and/or a gene for a ubiquitin peptidase as an active ingredient. Another embodiment provides a method of enhancing the efficacy of an anti-c-Met antibody including inhibiting a ubiquitin peptidase (e.g., USP8) and/or a gene for a ubiquitin peptidase. The step of inhibiting a ubiquitin peptidase and/or a gene for a ubiquitin peptidase may be performed by administering a pharmaceutical amount of an inhibitor against a ubiquitin peptidase (e.g., USP8) and/or a gene for a ubiquitin peptidase to a subject, or by mutating an active site of the ubiquitin peptidase or the gene for a ubiquitin peptidase. The subject may be one in need of treatment with an anti-c-Met antibody. Subjects may be mammals such as primates, including humans and monkeys, and rodents, including mice and rats, or cells or tissues isolated from the living body thereof. The subject may be a cancer patient, a cancer cell or a cancer tissue. Mutation in the active site of the ubiquitin peptidase or in the gene encoding the ubiquitin peptidase may be performed by substituting the amino acid residue Cys at 786^{th} position of USP8, for example human USP8 (NP_{—}001122082; SEQ ID NO: 109) with Ser (C786S), or substituting the USP8 gene so as to encode the USP8 mutant (SEQ ID NO: 111). The mutation of the protein or the gene may be performed by any ordinary method in the relevant art. As shown in Example 5 and FIG. 5, an increase of USP8 leads to increased stability of LRIG1, whereas a mutation in an active site of USP8 decreases the stability of LRIG1, consequently increasing the activity of an anti-c-Met antibody.

The enhancement of the efficacy of an anti-c-Met antibody may include not only the increase of the anti-c-Met antibody's efficacy, i.e., the enhancement of the efficacy of cell internalization and/or degradation of c-Met, but also the decrease of side effects such as agonism of an anti-c-Met antibody.

The inhibitor against a ubiquitin peptidase and/or a gene for ubiquitin peptidase is as described above. In one embodiment, the inhibition of a ubiquitin peptidase and/or a gene for ubiquitin peptidase can be performed by knock-down of the gene for ubiquitin peptidase (e.g., USP8) using at least one selected from the group consisting of a chemical drug, siRNA, microRNA, shRNA, and aptamer, or by substitution or deletion of the gene. The substitution of the gene may refer to a substitution of at least one nucleotide of the cDNA or mRNA with different nucleotides, and the deletion of the gene may refer to the removal of the gene or a part thereof. Both the substitution and deletion of the gene may lead to inhibition of expression of a ubiquitin peptidase having its intact function. For example, the substitution of the gene may be performed so that the gene encodes the USP8 mutant as described above. For example, the substitution of the gene may be performed so that the codon which originally encodes the amino acid residue Cys at 786^{th} position of human USP8 (NP_{—}001122082; SEQ ID NO: 109), encodes Ser instead.

Another embodiment provides a method for screening a (candidate) drug for preventing and/or treating a cancer using a ubiquitin peptidase and/or a ubiquitin peptidase coding gene.

The method for screening may include:

contacting (or treating) a candidate compound to a biological sample;

measuring a level of a ubiquitin peptidase and/or a ubiquitin peptidase coding gene in the biological sample; and

comparing the level of the ubiquitin peptidase and/or the ubiquitin peptidase coding gene in the biological sample contacted by (or treated with) the candidate compound to the level of the ubiquitin peptidase and/or the ubiquitin peptidase coding gene in a biological sample not contacted by (or treated) by the candidate compound.

The step of comparing may be performed by comparing the levels of the ubiquitin peptidase and/or the ubiquitin peptidase coding gene in the same biological sample measured before and after contact (or treatment) with the candidate compound, or by comparing the level of the ubiquitin peptidase and/or the ubiquitin peptidase coding gene in a part of the biological sample contacted by (or treated with) the candidate compound to that in other part of the biological sample not contacted by (or treated with) the candidate compound.

In cases where the level of the ubiquitin peptidase and/or the ubiquitin peptidase coding gene in the biological sample contacted by (or treated with) the candidate compound is decreased compared to that in the biological sample not contacted by (or treated with) with the candidate compound, that is, in cases where it is confirmed that the candidate inhibits the ubiquitin peptidase and/or the ubiquitin peptidase coding gene, the candidate compound may be determined as a (candidate) drug for preventing and/or treating a cancer.

The biological sample may be a cell or tissue separated from a living body of a mammal including human, and for example, it may be a cancer cell or a cancer tissue.

The candidate compound may be at least one selected from the group consisting of various kinds of compounds, for example, proteins, polypeptides, oligopeptides, polynucleotides, oligonucleotides, and other various chemical materials.

The step of measuring the level of the ubiquitin peptidase and/or the ubiquitin peptidase coding gene in the biological sample may be performed by a measurement using an ordinary means for detection and/or quantification of a protein or a gene, and/or by analysis of the measurement results. Means for detection and/or quantification of a protein or a gene are as described above.

A drug for preventing and/or treating a cancer screened by the above method may exhibit an increased synergistic effect by being co-administered with an anti-c-Met antibody. In addition, due to the synergistic effect, it is possible to reduce the administration amount of the anti-c-Met antibody, thereby decreasing side effects thereof. Therefore, the drug for preventing and/or treating a cancer screened by the above method is a good partner drug for co-administration with an anti-c-Met antibody, that is, the drug is suitable for use in a combination therapy using an anti-c-Met antibody.

In an embodiment, the anti c-Met antibody may be any antibody and/or antigen-binding fragment thereof, which recognizes c-Met protein as an antigen. In particular, the anti c-Met antibody may recognize a specific region of c-Met, e.g., a specific region in the SEMA domain, as an epitope. It may be any antibody or antigen-binding fragment that acts on c-Met to induce c-Met intracellular internalization and degradation.

As used herein, unless otherwise stated, the term “anti-c-Met antibody” may be used to include not only an complete antibody but also an antigen-binding fragment thereof.

The term “c-Met” or “c-Met protein” refers to a receptor tyrosine kinase (RTK) which binds hepatocyte growth factor (HGF). c-Met may be a c-Met protein from any species, particularly a mammal, for instance, primates such as human c-Met (e.g., NP_{—}000236) or monkey c-Met (e.g., Macaca mulatta, NP_{—}001162100), or rodents such as mouse c-Met (e.g., NP_{—}032617.2) or rat c-Met (e.g., NP_{—}113705.1). The c-Met protein may include a polypeptide encoded by the nucleotide sequence identified as GenBank Accession Number NM_{—}000245, a polypeptide having the amino acid sequence identified as GenBank Accession Number NP_{—}000236 or extracellular domains thereof. The receptor tyrosine kinase c-Met participates in various mechanisms, such as cancer development, metastasis, migration of cancer cell, invasion of cancer cell, and angiogenesis.

c-Met, a receptor for hepatocyte growth factor (HGF) may be divided into three portions: extracellular, transmembrane, and intracellular. The extracellular portion is composed of an a (alpha)-subunit and a β (beta)-subunit which are linked to each other through a disulfide bond, and contains a SEMA domain responsible for binding HGF, a PSI domain (plexin-semaphorins-integrin homology domain) and an IPT domain (immunoglobulin-like fold shared by plexins and transcriptional factors domain). The SEMA domain of c-Met protein may have the amino acid sequence of SEQ ID NO: 79, and is an extracellular domain that functions to bind HGF. A specific region of the SEMA domain, that is, a region having the amino acid sequence of SEQ ID NO: 71, which corresponds to a range from amino acid residues 106 to 124 of the amino acid sequence of the SEMA domain (SEQ ID NO: 79) of c-Met protein, is a loop region between the second and the third propellers within the epitopes of the SEMA domain. The region acts as an epitope for the specific anti-c-Met antibody of the present disclosure.

The term “epitope” as used herein, refers to an antigenic determinant, a part of an antigen recognized by an antibody. In one embodiment, the epitope may be a region comprising 5 or more contiguous (consecutive or non-consecutive) amino acid residues within the SEMA domain (SEQ ID NO: 79) of c-Met protein, for instance, 5 to 19 contiguous amino acid residues within the amino acid sequence of SEQ ID NO: 71. For example, the epitope may be a polypeptide having 5 to 19 contiguous amino acids selected from among partial combinations of the amino acid sequence of SEQ ID NO: 71, wherein the polypeptide essentially includes the amino sequence of SEQ ID NO: 73 (EEPSQ) serving as an essential element for the epitope. For example, the epitope may be a polypeptide comprising, consisting essentially of, or consisting of the amino acid sequence of SEQ ID NO: 71, SEQ ID NO: 72, or SEQ ID NO: 73.

The epitope having the amino acid sequence of SEQ ID NO: 72 corresponds to the outermost part of the loop between the second and third propellers within the SEMA domain of a c-Met protein. The epitope having the amino acid sequence of SEQ ID NO: 73 is a site to which the antibody or antigen-binding fragment according to one embodiment most specifically binds.

Thus, the anti-c-Met antibody may specifically bind to an epitope which has 5 to 19 contiguous amino acids selected from the amino acid sequence of SEQ ID NO: 71, including SEQ ID NO: 73 (EEPSQ) as an essential element. For example, the anti-c-Met antibody may specifically bind to an epitope including the amino acid sequence of SEQ ID NO: 71, SEQ ID NO: 72, or SEQ ID NO: 73.

In one embodiment, the anti-c-Met antibody or an antigen-binding fragment thereof may comprise or consist essentially of:

at least one heavy chain complementarity determining region (CDR) selected from the group consisting of (a) a CDR-H1 comprising the amino acid sequence of SEQ ID NO: 4; (b) a CDR-H2 comprising the amino acid sequence of SEQ ID NO: 5, SEQ ID NO: 2, or an amino acid sequence comprising 8-19 consecutive amino acids within SEQ ID NO: 2 including amino acid residues from the 3^{rd} to 10^{th} positions of SEQ ID NO: 2; and (c) a CDR-H3 comprising the amino acid sequence of SEQ ID NO: 6, SEQ ID NO: 85, or an amino acid sequence comprising 6-13 consecutive amino acids within SEQ ID NO: 85 including amino acid residues from the 1^{st} to 6^{th} positions of SEQ ID NO: 85, or a heavy chain variable region comprising the at least one heavy chain complementarity determining region;

at least one light chain complementarity determining region (CDR) selected from the group consisting of (a) a CDR-L1 comprising the amino acid sequence of SEQ ID NO: 7, (b) a CDR-L2 comprising the amino acid sequence of SEQ ID NO: 8, and (c) a CDR-L3 comprising the amino acid sequence of SEQ ID NO: 9, SEQ ID NO: 86, or an amino acid sequence comprising 9-17 consecutive amino acids within SEQ ID NO: 89 including amino acid residues from the 1^{st} to 9^{th} positions of SEQ ID NO: 89, or a light chain variable region comprising the at least one light chain complementarity determining region;

a combination of the at least one heavy chain complementarity determining region and at least one light chain complementarity determining region; or

a combination of the heavy chain variable region and the light chain variable region.

Herein, the amino acid sequences of SEQ ID NOS: 4 to 9 are respectively represented by following Formulas I to VI, below:

Xaa₁-Xaa₂-Tyr-Tyr-Met-Ser(SEQ ID NO: 4), Formula I

wherein Xaa₁ is absent or Pro or Ser, and Xaa₂ is Glu or Asp,

Arg-Asn-Xaa₃-Xaa₄-Asn-Gly-Xaa₅-Thr(SEQ ID NO: 5), Formula II

wherein Xaa₃ is Asn or Lys, Xaa₄ is Ala or Val, and Xaa₅ is Asn or Thr,

Asp-Asn-Trp-Leu-Xaa₆-Tyr(SEQ ID NO: 6), Formula III

wherein Xaa₆ is Ser or Thr,

Lys-Ser-Ser-Xaa₇-Ser-Leu-Leu-Ala-Xaa₈-Gly-Asn-Xaa₉-Xaa₁₀-Asn-Tyr-Leu-Ala(SEQ ID NO: 7) Formula IV

wherein Xaa₇ is His, Arg, Gln, or Lys, Xaa₈ is Ser or Trp, Xaa₉ is His or Gln, and Xaa₁₀ is Lys or Asn,

Trp-Xaa₁₁-Ser-Xaa₁₂-Arg-Val-Xaa₁₃(SEQ ID NO: 8) Formula V

wherein Xₐₐ₁₁ is Ala or Gly, Xaa₁₂ is Thr or Lys, and Xaa₁₃ is Ser or Pro, and

Xaa₁₄-Gln-Ser-Tyr-Ser-Xaa₁₅-Pro-Xaa₁₆-Thr(SEQ ID NO: 9) Formula VI

wherein Xaa₁₄ is Gly, Ala, or Gln, Xaa₁₅ is Arg, His, Ser, Ala, Gly, or Lys, and Xaa₁₆ is Leu, Tyr, Phe, or Met.

In one embodiment, the CDR-H1 may comprise or consist essentially of an amino acid sequence selected from the group consisting of SEQ ID NOS: 1, 22, 23, and 24. The CDR-H2 may comprise or consist essentially of an amino acid sequence selected from the group consisting of SEQ ID NOS: 2, 25, and 26. The CDR-H3 may comprise or consist essentially of an amino acid sequence selected from the group consisting of SEQ ID NOS: 3, 27, 28, and 85.

The CDR-L1 may comprise or consist essentially of an amino acid sequence selected from the group consisting of SEQ ID NOS: 10, 29, 30, 31, 32, 33, and 106. The CDR-L2 may comprise or consist essentially of an amino acid sequence selected from the group consisting of SEQ ID NOS: 11, 34, 35, and 36. The CDR-L3 may comprise or consist essentially of an amino acid sequence selected from the group consisting of SEQ ID NOS: 12, 13, 14, 15, 16, 37, 86, and 89.

In another embodiment, the antibody or antigen-binding fragment may include a heavy chain variable region comprising a polypeptide (CDR-H1) including an amino acid sequence selected from the group consisting of SEQ ID NOS: 1, 22, 23, and 24, a polypeptide (CDR-H2) including an amino acid sequence selected from the group consisting of SEQ ID NOS: 2, 25, and 26, and a polypeptide (CDR-H3) including an amino acid sequence selected from the group consisting of SEQ ID NOS: 3, 27, 28, and 85; and a light chain variable region comprising a polypeptide (CDR-L1) including an amino acid sequence selected from the group consisting of SEQ ID NOS: 10, 29, 30, 31, 32, 33 and 106, a polypeptide (CDR-L2) including an amino acid sequence selected from the group consisting of SEQ ID NOS: 11, 34, 35, and 36, and a polypeptide (CDR-L3) including an amino acid sequence selected from the group consisting of SEQ ID NOS 12, 13, 14, 15, 16, 37, 86, and 89.

In one embodiment of the anti-c-Met antibody or antigen-binding fragment, the variable region of the heavy chain includes the amino acid sequence of SEQ ID NO: 17, 74, 87, 90, 91, 92, 93, or 94 and the variable region of the light chain includes the amino acid sequence of SEQ ID NO: 18, 19, 20, 21, 75, 88, 95, 96, 97, 98, 99, or 107.

Animal-derived antibodies produced by immunizing non-immune animals with a desired antigen generally invoke immunogenicity when injected to humans for the purpose of medical treatment, and thus chimeric antibodies have been developed to inhibit such immunogenicity. Chimeric antibodies are prepared by replacing constant regions of animal-derived antibodies that cause an anti-isotype response with constant regions of human antibodies by genetic engineering. Chimeric antibodies are considerably improved in terms of anti-isotype response compared to animal-derived antibodies, but animal-derived amino acids still have variable regions, so that chimeric antibodies have side effects with respect to a potential anti-idiotype response. Humanized antibodies have been developed to reduce such side effects. Humanized antibodies are produced by grafting complementarity determining regions (CDR) which serve an important role in antigen binding in variable regions of chimeric antibodies into a human antibody framework.

In using CDR grafting to produce humanized antibodies, choosing which optimized human antibodies to use for accepting CDRs of animal-derived antibodies is critical. Antibody databases, analysis of a crystal structure, and technology for molecule modeling are used. However, even when the CDRs of animal-derived antibodies are grafted to the most optimized human antibody framework, amino acids positioned in a framework of the animal-derived CDRs affecting antigen binding are present. Therefore, in many cases, antigen binding affinity is not maintained, and thus application of additional antibody engineering technology for recovering the antigen binding affinity is necessary.

The anti c-Met antibodies may be, but are not limited to, animal antibodies (e.g., mouse-derived antibodies), chimeric antibodies (e.g., mouse-human chimeric antibodies), humanized antibodies, or human antibodies. The antibodies or antigen-binding fragments thereof may be isolated from a living body or non-naturally occurring. The antibodies or antigen-binding fragments thereof may be synthetic or recombinant.

An intact antibody includes two full-length light chains and two full-length heavy chains, in which each light chain is linked to a heavy chain by disulfide bonds. The antibody has a heavy chain constant region and a light chain constant region. The heavy chain constant region is of a gamma (γ), mu (μ), alpha (α), delta (δ), or epsilon (ε) type, which may be further categorized as gamma 1 (γ1), gamma 2(γ2), gamma 3(γ3), gamma 4(γ4), alpha 1(α1), or alpha 2(α2). The light chain constant region is of either a kappa (κ) or lambda (λ) type.

As used herein, the term “heavy chain” refers to full-length heavy chain, and fragments thereof, including a variable region V_{H} that includes amino acid sequences sufficient to provide specificity to antigens, and three constant regions, C_{H1}, C_{H2}, and C_{H3}, and a hinge. The term “light chain” refers to a full-length light chain and fragments thereof, including a variable region V_{L} that includes amino acid sequences sufficient to provide specificity to antigens, and a constant region C_{L}.

The term “complementarity determining region (CDR)” refers to an amino acid sequence found in a hyper variable region of a heavy chain or a light chain of immunoglobulin. The heavy and light chains may respectively include three CDRs (CDRH1, CDRH2, and CDRH3; and CDRL1, CDRL2, and CDRL3). The CDR may provide contact residues that play an important role in the binding of antibodies to antigens or epitopes. The terms “specifically binding” and “specifically recognized” are well known to one of ordinary skill in the art, and indicate that an antibody and an antigen specifically interact with each other to lead to an immunological activity.

The term “antigen-binding fragment” used herein refers to fragments of an intact immunoglobulin including portions of a polypeptide including antigen-binding regions having the ability to specifically bind to the antigen. In a particular embodiment, the antigen-binding fragment may be scFv, (scFv)₂, scFvFc, Fab, Fab′, or F(ab′)₂, but is not limited thereto.

Among the antigen-binding fragments, Fab that includes light chain and heavy chain variable regions, a light chain constant region, and a first heavy chain constant region C_{H1}, has one antigen-binding site.

The Fab′ fragment is different from the Fab fragment, in that Fab′ includes a hinge region with at least one cysteine residue at the C-terminal of C_{H1}.

The F(ab′)₂ antibody is formed through disulfide bridging of the cysteine residues in the hinge region of the Fab′ fragment.

Fv is the smallest antibody fragment with only a heavy chain variable region and a light chain variable region. Recombination techniques of generating the Fv fragment are widely known in the art.

Two-chain Fv includes a heavy chain variable region and a light chain region which are linked by a non-covalent bond. Single-chain Fv generally includes a heavy chain variable region and a light chain variable region which are linked by a covalent bond via a peptide linker or linked at the C-terminals to have a dimer structure like the two-chain Fv. The peptide linker may be the same as described above, including, but not limited to, those having an amino acid length of 1 to 100, 2 to 50, particularly 5 to 25, and any kinds of amino acids may be included without any restrictions.

The antigen-binding fragments may be obtained using protease (for example, the Fab fragment may be obtained by restricted cleavage of a whole antibody with papain, and the F(ab′)₂ fragment may be obtained by cleavage with pepsin), or may be prepared by using a genetic recombination technique.

The term “hinge region,” as used herein, refers to a region between CH1 and CH2 domains within the heavy chain of an antibody which functions to provide flexibility for the antigen-binding site.

When an animal antibody undergoes a chimerization process, the IgG1 hinge of animal origin is replaced with a human IgG1 hinge or IgG2 hinge while the disulfide bridges between two heavy chains are reduced from three to two in number. In addition, an animal-derived IgG1 hinge is shorter than a human IgG1 hinge. Accordingly, the rigidity of the hinge is changed. Thus, a modification of the hinge region may bring about an improvement in the antigen binding efficiency of the humanized antibody. The modification of the hinge region through amino acid deletion, addition, or substitution is well-known to those skilled in the art.

In one embodiment, the anti-c-Met antibody or an antigen-binding fragment thereof may be modified by any combination of deletion, insertion, addition, or substitution of at least one amino acid residue on the amino acid sequence of the hinge region so that it exhibit enhanced antigen-binding efficiency. For example, the antibody may include a hinge region including the amino acid sequence of SEQ ID NO: 100(U7-HC6), 101(U6-HC7), 102(U3-HC9), 103(U6-HC8), or 104(U8-HC5), or a hinge region including the amino acid sequence of SEQ ID NO: 105 (non-modified human hinge). In particular, the hinge region has the amino acid sequence of SEQ ID NO: 100 or 101.

In one embodiment, the anti-c-Met antibody may be a monoclonal antibody. The monoclonal antibody may be produced by the hybridoma cell line deposited with Accession No. KCLRF-BP-00220, which binds specifically to the extracellular region of c-Met protein (refer to Korean Patent Publication No. 2011-0047698, the disclosure of which is incorporated in its entirety herein by reference). The anti-c-Met antibody may include all the antibodies defined in Korean Patent Publication No. 2011-0047698.

In the anti-c-Met antibody, the rest of the portion of the light chain and the heavy chain portion except the CDRs, the light chain variable region, and the heavy chain variable region as defined above, for example, the light chain constant region and the heavy chain constant region, may be those from any subtype of immunoglobulin (e.g., IgA, IgD, IgE, IgG (IgG1, IgG2, IgG3, IgG4), IgM, and the like).

By way of further example, the anti-c-Met antibody or the antibody fragment may include:

a heavy chain including the amino acid sequence selected from the group consisting of the amino acid sequence of SEQ ID NO: 62 (wherein the amino acid sequence from amino acid residues from the 1^{st} to 17^{th} positions is a signal peptide), or the amino acid sequence from the 18^{th} to 462^{nd} positions of SEQ ID NO: 62, the amino acid sequence of SEQ ID NO: 64 (wherein the amino acid sequence from the 1^{st} to 17^{th} positions is a signal peptide), the amino acid sequence from the 18^{th} to 461^{st} positions of SEQ ID NO: 64, the amino acid sequence of SEQ ID NO: 66 (wherein the amino acid sequence from the 1^{st} to 17^{th} positions is a signal peptide), and the amino acid sequence from the 18^{th} to 460^{th} positions of SEQ ID NO: 66; and

a light chain including the amino acid sequence selected from the group consisting of the amino acid sequence of SEQ ID NO: 68 (wherein the amino acid sequence from the 1^{st} to 20^{th} positions is a signal peptide), the amino acid sequence from the 21^{st} to 240^{th} positions of SEQ ID NO: 68, the amino acid sequence of SEQ ID NO: 70 (wherein the amino acid sequence from the 1^{st} to 20^{th} positions is a signal peptide), the amino acid sequence from the 21^{st} to 240^{th} positions of SEQ ID NO: 70, and the amino acid sequence of SEQ ID NO: 108.

For example, the anti-c-Met antibody may be selected from the group consisting of:

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 68 or the amino acid sequence from the 21^{st} to 240^{th} positions of SEQ ID NO: 68;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 68 or the amino acid sequence from the 21^{st} to 240^{th} positions of SEQ ID NO: 68;

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 68 or the amino acid sequence from the 21^{st} to 240^{th} positions of SEQ ID NO: 68;

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 70 or the amino acid sequence from the 21^{st} to 240^{th} positions of SEQ ID NO: 70;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 70 or the amino acid sequence from the 21^{st} to 240^{th} positions of SEQ ID NO: 70;

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 70 or the amino acid sequence from the 21^{st} to 240^{th} positions of SEQ ID NO: 70;

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 108;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 108; and

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 108.

According to an embodiment, the anti-c-Met antibody may include a heavy chain including the amino acid sequence from the 18^{th} to 460^{th} positions of SEQ ID NO: 66 and a light chain including the sequence from the 21^{st} to 240^{th} positions of SEQ ID NO: 68, or a heavy chain including the amino acid sequence from the 18^{th} to 460^{th} positions of SEQ ID NO: 66 and a light chain including the sequence of SEQ ID NO: 108.

The polypeptide of SEQ ID NO: 70 is a light chain including human kappa (K) constant region, and the polypeptide with the amino acid sequence of SEQ ID NO: 68 is a polypeptide obtained by replacing histidine at position 62 (corresponding to position 36 of SEQ ID NO: 68 according to kabat numbering) of the polypeptide with the amino acid sequence of SEQ ID NO: 70 with tyrosine. The production yield of the antibodies may be increased by the replacement. The polypeptide with the amino acid sequence of SEQ ID NO: 108 is a polypeptide obtained by replacing serine at position 32 (position 27e according to kabat numbering in the amino acid sequence from amino acid residues 21 to 240 of SEQ ID NO: 68; positioned within CDR-L1) with tryptophan. By such replacement, antibodies and antibody fragments including such sequences exhibits increased activities, such as c-Met biding affinity, c-Met degradation activity, and Akt phosphorylation inhibition.

In another embodiment, the anti-c-Met antibody may include a light chain complementarity determining region including the amino acid sequence of SEQ ID NO: 106, a light chain variable region including the amino acid sequence of SEQ ID NO: 107, or a light chain including the amino acid sequence of SEQ ID NO: 108.

The mixture wherein a pharmaceutically effective amount of an anti-c-Met antibody or an antigen-binding fragment thereof and a pharmaceutically effective amount of an inhibitor against a ubiquitin peptidase and/or a ubiquitin peptidase coding gene are mixed, the first pharmaceutical composition containing a pharmaceutically effective amount of an anti-c-Met antibody or an antigen-binding fragment thereof as an active ingredient and the second pharmaceutical composition containing a pharmaceutically effective amount an inhibitor against a ubiquitin peptidase and/or a ubiquitin peptidase coding gene as an active ingredient, may be provided (or administered) along with a pharmaceutically acceptable carrier, diluent, and/or excipient.

The pharmaceutically acceptable carrier to be included in the mixture or the pharmaceutical composition may be those commonly used for the formulation of antibodies, which may be one or more selected from the group consisting of lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and mineral oil, but are not limited thereto. The pharmaceutical composition may further include one or more selected from the group consisting of a lubricant, a wetting agent, a sweetener, a flavor enhancer, an emulsifying agent, a suspension agent, and preservative.

The pharmaceutical composition, the mixture, or each active ingredient may be administered orally or parenterally. The parenteral administration may include intravenous injection, subcutaneous injection, muscular injection, intraperitoneal injection, endothelial administration, local administration, intranasal administration, intrapulmonary administration, and rectal administration. Since oral administration leads to digestion of proteins or peptides, an active ingredient in the compositions for oral administration must be coated or formulated to prevent digestion in the stomach. In addition, the compositions may be administered using an optional device that enables an active substance to be delivered to target cells.

The term “the pharmaceutically effective amount” as used in this specification refers to an amount at which each active ingredient can exert pharmaceutically significant effects.

For one-time administration, a pharmaceutically effective amount of an anti-c-Met antibody or an antigen-binding fragment thereof and a pharmaceutically effective amount of an inhibitor against the target substance may be prescribed in a variety way, depending on many factors including formulation methods, administration manners, ages of patients, body weight, gender, pathologic conditions, diets, administration time, administration interval, administration route, excretion speed, and reaction sensitivity. For example, the effective amount of the inhibitor against the target substance (e.g., a ubiquitin peptidase and/or its coding gene) may be, but not limited to, in ranges of 0.001 to 100 mg/kg, or 0.02 to 10 mg/kg for one-time administration and the effective amount of the anti-c-Met antibodies or antigen binding fragments thereof may be, but not limited to, in ranges of 0.001 to 100 mg/kg, or 0.02 to 10 mg/kg for their one-time administration.

The effective amount for one-time administration may be formulated into a single formulation in a unit dosage form or formulated in suitably divided dosage forms, or it may be manufactured to be contained in a multiple dosage container. For the kit, the effective amount of the inhibitor against the target substance and the effective amount of the anti-c-Met antibodies or antigen binding fragments thereof for one-time administration (single dose) may be contained in a package container as a base unit.

The administration interval between the administrations is defined as a period between the first administration and the following administration. The administration interval may be, but is not limited to, 24 hours to 30 days (e.g., 10 hours, 15 hours, 20 hours, 1 day, 2 days, 3 days, 4 days, 5 days, 6, days, 7 days, 10 days, 14 days, 21 days, or 28 days) and particularly 7 to 14 days or so. For the combined therapy, the first pharmaceutical composition containing a pharmaceutically effective amount of an anti-c-Met antibody or an antigen-binding fragment thereof as an active ingredient, and the second pharmaceutical composition containing a pharmaceutically effective amount of an inhibitor against a ubiquitin peptidase and/or a ubiquitin peptidase coding gene as an active ingredient may be co-administered in a given time interval (e.g., several minutes, several hours or several days, or several weeks) to be determined by considerations like the type of disease, and a patient's condition. For example, the first pharmaceutical composition and the second pharmaceutical composition may be simultaneously administered (administration interval within 1 minute) or sequentially administered (administration interval of 1 minute or over), and in case of sequential administration, the administration interval between the first pharmaceutical composition and the second pharmaceutical composition may be 1 to 60 minutes, particularly, 1 minute to 10 minutes, and they may be administered in any order.

The combined mixture or the pharmaceutical compositions may be a solution in oil or an aqueous medium, a suspension, a syrup, an emulsifying solution form, or they may be formulated into a form of an extract, elixirs, powders, granules, a tablet or a capsule, and they may further include a dispersing agent or a stabilizing agent in their formulation.

The pharmaceutical composition containing the anti-c-Met antibody or antigen binding fragments thereof may be formulated into an immunoliposome since it contains an antibody or an antigen binding fragment. A liposome containing an antibody may be prepared using any methods well known in the pertinent field. The immunoliposome may be a lipid composition including phosphatidylcholine, cholesterol, and polyethyleneglycol-derived phosphatidylethanolamine, and may be prepared by a reverse phase evaporation method. For example, Fab′ fragments of an antibody may be conjugated to the liposome through a disulfide-exchange reaction. A chemical drug, such as doxorubicin, may further be included in the liposome.

The pharmaceutical compositions or the method may be used for the prevention and/or treatment of a cancer. The cancer may be associated with overexpression and/or (abnormal) activation of c-Met. The cancer may be a solid cancer or a blood cancer. For example, the cancer may be at least one selected from the group consisting of squamous cell carcinoma, small-cell lung cancer, non-small-cell lung cancer, adenocarcinoma of the lung, squamous cell carcinoma of the lung, peritoneal carcinoma, skin cancer, melanoma in the skin or eyeball, rectal cancer, cancer near the anus, esophagus cancer, small intestinal tumor, endocrine gland cancer, parathyroid cancer, adrenal cancer, soft-tissue sarcoma, urethral cancer, chronic or acute leukemia, lymphocytic lymphoma, hepatoma, gastrointestinal cancer, gastric cancer, pancreatic cancer, glioblastoma, cervical cancer, ovarian cancer, liver cancer, bladder cancer, hepatocellular adenoma, breast cancer, colon cancer, large intestine cancer, endometrial carcinoma or uterine carcinoma, salivary gland tumor, kidney cancer, prostate cancer, vulvar cancer, thyroid cancer, head and neck cancers, osteosarcoma, and brain cancer, but is not limited thereto.

The prevention and/or treatment effects of the cancers may include effects of not only suppressing the growth of the cancer cells but also suppressing progression of cancers due to migration, invasion, and metastasis thereof. Therefore, the cancers curable by the combined therapy of the disclosure include both primary cancers and metastatic cancers. The aforementioned activities of a ubiquitin peptidase (e.g., USP8) and/or a gene encoding the same may be extended to HGF/c-Met inhibitors as well as anti-c-Met antibodies.

As described above, the finding of the use of a ubiquitin peptidase (e.g., USP8) and/or a gene encoding the same as a biomarker for HGF/c-Met inhibitor such as anti-c-Met antibodies can lead to the following effects: the efficacy of an anti-c-Met antibody can be improved by knock-down of USP8 in patients having high level of a ubiquitin peptidase (e.g., USP8) and/or a gene encoding the same, and agonism of anti-c-Met antibodies can be prevented by selecting patients having high level of LRIG1 and low level of USP8.

The present invention can be applied to diseases which are associated with c-Met/HGF signal transduction pathway and ubiquitination pathway, other than cancers.

### EXAMPLES

Hereafter, the present invention will be described in detail by examples.

The following examples are intended merely to illustrate the invention and are not construed to restrict the invention.

### Reference Example 1

### Construction of Anti-c-Met Antibody

1.1. Production of “AbF46”, a Mouse Antibody to c-Met

1.1.1. Immunization of Mouse

To obtain immunized mice necessary for the development of a hybridoma cell line, each of five BALB/c mice (Japan SLC, Inc.), 4 to 6 weeks old, was intraperitoneally injected with a mixture of 100 μg of human c-Met/Fc fusion protein (R&D Systems) and one volume of complete Freund's adjuvant. Two weeks after the injection, a second intraperitoneal injection was conducted on the same mice with a mixture of 50 μg of human c-Met/Fc protein and one volume of incomplete Freund's adjuvant. One week after the second immunization, the immune response was finally boosted. Three days later, blood was taken from the tails of the mice and the sera were 1/1000 diluted in PBS and used to examine a titer of antibody to c-Met by ELISA. Mice found to have a sufficient antibody titer were selected for use in the cell fusion process.

1.1.2. Cell Fusion and Production of Hybridoma

Three days before cell fusion, BALB/c mice (Japan SLC, Inc.) were immunized with an intraperitoneal injection of a mixture of 50 μg of human c-Met/Fc fusion protein and one volume of PBS. The immunized mice were anesthetized before excising the spleen from the left half of the body. The spleen was meshed to separate splenocytes which were then suspended in a culture medium (DMEM, GIBCO, Invitrogen). The cell suspension was centrifuged to recover the cell layer. The splenocytes thus obtained (1×10⁸ cells) were mixed with myeloma cells (Sp2/0) (1×10⁸ cells), followed by spinning to give a cell pellet. The cell pellet was slowly suspended, treated with 45% polyethylene glycol (PEG) (1 mL) in DMEM for 1 min at 37° C., and supplemented with 1 mL of DMEM. To the cells was added 10 mL of DMEM over 10 min, after which incubation was conducted in a water bath at 37° C. for 5 min. Then the cell volume was adjusted to 50 mL before centrifugation. The cell pellet thus formed was resuspended at a density of 1˜2×10⁵ cells/mL in a selection medium (HAT medium) and 0.1 mL of the cell suspension was allocated to each well of 96-well plates which were then incubated at 37° C. in a CO₂ incubator to establish a hybridoma cell population.

1.1.3. Selection of Hybridoma Cells Producing Monoclonal Antibodies to c-Met Protein

From the hybridoma cell population established in Reference Example 1.1.2, hybridoma cells which showed a specific response to c-Met protein were screened by ELISA using human c-Met/Fc fusion protein and human Fc protein as antigens.

Human c-Met/Fc fusion protein was seeded in an amount of 50 μL (2 μg/mL)/well to microtiter plates and allowed to adhere to the surface of each well. The antibody that remained unbound was removed by washing. For use in selecting the antibodies that do not bind c-Met but recognize Fc, human Fc protein was attached to the plate surface in the same manner.

The hybridoma cell culture obtained in Reference Example 1.1.2 was added in an amount of 50 μL to each well of the plates and incubated for 1 hour. The cells remaining unreacted were washed out with a sufficient amount of Tris-buffered saline and Tween 20 (TBST). Goat anti-mouse IgG-horseradish peroxidase (HRP) was added to the plates and incubated for 1 hour at room temperature. The plates were washed with a sufficient amount of TBST, followed by reacting the peroxidase with a substrate (OPD). Absorbance at 450 nm was measured on an ELISA reader.

Hybridoma cell lines which secrete antibodies that specifically and strongly bind to human c-Met but not human Fc were selected repeatedly. From the hybridoma cell lines obtained by repeated selection, a single clone producing a monoclonal antibody was finally separated by limiting dilution. The single clone of the hybridoma cell line producing the monoclonal antibody was deposited with the Korean Cell Line Research Foundation, an international depository authority located at Yungun-Dong, Jongno-Gu, Seoul, Korea, on Oct. 6, 2009, with Accession No. KCLRF-BP-00220 according to the Budapest Treaty (refer to Korean Patent Laid-Open Publication No. 2011-0047698).

1.1.4. Production and Purification of Monoclonal Antibody

The hybridoma cell line obtained in Reference Example 1.1.3 was cultured in a serum-free medium, and the monoclonal antibody (AbF46) was produced and purified from the cell culture.

First, the hybridoma cells cultured in 50 mL of a medium (DMEM) supplemented with 10% (v/v) FBS were centrifuged and the cell pellet was washed twice or more with 20 mL of PBS to remove the FBS therefrom. Then, the cells were resuspended in 50 mL of DMEM and incubated for 3 days at 37° C. in a CO₂ incubator.

After the cells were removed by centrifugation, the supernatant was stored at 4° C. before use or immediately used for the separation and purification of the antibody. An AKTA system (GE Healthcare) equipped with an affinity column (Protein G agarose column; Pharmacia, USA) was used to purify the antibody from 50 to 300 mL of the supernatant, followed by concentration with a filter (Amicon). The antibody in PBS was stored before use in the following examples.

1.2. Construction of chAbF46, a Chimeric Antibody to c-Met

A mouse antibody is apt to elicit immunogenicity in humans. To solve this problem, chAbF46, a chimeric antibody, was constructed from the mouse antibody AbF46 produced in Experimental Example 1.1.4 by replacing the constant region, but not the variable region responsible for antibody specificity, with an amino sequence of the human IgG1 antibody.

In this regard, a gene was designed to include the nucleotide sequence of “EcoRI-signal sequence-VH-NheI-CH-TGA-XhoI” (SEQ ID NO: 38) for a heavy chain and the nucleotide sequence of “EcoRI-signal sequence-VL-BsiWI-CL-TGA-XhoI” (SEQ ID NO: 39) for a light chain and synthesized. Then, a DNA fragment having the heavy chain nucleotide sequence (SEQ ID NO: 38) and a DNA fragment having the light chain nucleotide sequence (SEQ ID NO: 39) were digested with EcoRI (NEB, R0101S) and XhoI (NEB, R0146S) before cloning into a pOptiVEC™-TOPO TA Cloning Kit enclosed in an OptiCHO™ Antibody Express Kit (Cat no. 12762-019, Invitrogen), and a pcDNA™3.3-TOPO TA Cloning Kit (Cat no. 8300-01), respectively.

Each of the constructed vectors was amplified using Qiagen Maxiprep kit (Cat no. 12662), and a transient expression was performed using Freestyle™ MAX 293 Expression System (invitrogen). 293 F cells were used for the expression and cultured in FreeStyle™ 293 Expression Medium in a suspension culture manner. At one day before the transient expression, the cells were provided in the concentration of 5×10⁵ cells/ml, and after 24 hours, when the cell number reached 1×10⁶ cells/ml, the transient expression was performed. A transfection was performed by a liposomal reagent method using Freestyle™ MAX reagent (invitrogen), wherein in a 15 ml tube, the DNA was provided in the mixture ratio of 1:1 (heavy chain DNA: light chain DNA) and mixed with 2 ml of OptiPro™ SFM (invtrogen) (tube A), and in another 15 ml tube, 100 ul (microliter) of Freestyle™ MAX reagent and 2 ml of OptiPro™ SFM were mixed (tube B), followed by mixing tube A and tube B and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

Afterwards, the cells were incubated in DMEM supplemented with 10% (v/v) FBS for 5 hours at 37° C. under a 5% CO₂ condition and then in FBS-free DMEM for 48 hours at 37° C. under a 5% CO₂ condition.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE healthcare, 17-0405-03), followed by elution with an IgG elution buffer (Thermo Scientific, 21004). The buffer was exchanged with PBS to purify a chimeric antibody AbF46 (hereinafter referred to as “chAbF46”).

1.3. Construction of Humanized Antibody huAbF46 from Chimeric Antibody chAbF46

1.3.1. Heavy Chain Humanization

To design two domains H1-heavy and H3-heavy, human germline genes which share the highest identity/homology with the VH gene of the mouse antibody AbF46 purified in Reference Example 1.2 were analyzed. An Ig BLAST (www.ncbi.nlm.nih.gov/igblast/) result revealed that VH3-71 has an identity/identity/homology of 83% at the amino acid level. CDR-H1, CDR-H2, and CDR-H3 of the mouse antibody AbF46 were defined according to Kabat numbering. A design was made to introduce the CDR of the mouse antibody AbF46 into the framework of VH3-71. Hereupon, back mutations to the amino acid sequence of the mouse AbF46 were conducted at positions 30 (S→T), 48 (V→L), 73 (D→N), and 78 (T→L). Then, H1 was further mutated at positions 83 (R→K) and 84 (A→T) to finally establish H1-heavy (SEQ ID NO: 40) and H3-heavy (SEQ ID NO: 41).

For use in designing H4-heavy, human antibody frameworks were analyzed by a BLAST search. The result revealed that the VH3 subtype, known to be most stable, is very similar in framework and sequence to the mouse antibody AbF46. CDR-H1, CDR-H2, and CDR-H3 of the mouse antibody AbF46 were defined according to Kabat numbering and introduced into the VH3 subtype to construct H4-heavy (SEQ ID NO: 42).

1.3.2. Light Chain Humanization

To design two domains H1-light (SEQ ID NO: 43) and H2-light (SEQ ID NO: 44), human germline genes which share the highest identity/homology with the VH gene of the mouse antibody AbF46 were analyzed. An Ig BLAST search result revealed that VK4-1 has a identity/homology of 75% at the amino acid level. CDR-L1, CDR-L2, and CDR-L3 of the mouse antibody AbF46 were defined according to Kabat numbering. A design was made to introduce the CDR of the mouse antibody AbF46 into the framework of VK4-1. Hereupon, back mutations to the amino acid sequence of the mouse AbF46 were conducted at positions 36 (Y→H), 46 (L→M), and 49 (Y→I). Only one back mutation was conducted at position 49 (Y→I) on H2-light.

To design H3-light (SEQ ID NO: 45), human germline genes which share the highest identity/homology with the VL gene of the mouse antibody AbF46 were analyzed by a search for BLAST. As a result, VK2-40 was selected. VL and VK2-40 of the mouse antibody AbF46 were found to have a identity/homology of 61% at an amino acid level. CDR-L1, CDR-L2, and CDR-L3 of the mouse antibody were defined according to Kabat numbering and introduced into the framework of VK4-1. Back mutations were conducted at positions 36 (Y→H), 46 (L→M), and 49 (Y→I) on H3-light.

For use in designing H4-light (SEQ ID NO: 46), human antibody frameworks were analyzed. A Blast search revealed that the Vk1 subtype, known to be the most stable, is very similar in framework and sequence to the mouse antibody AbF46. CDR-L1, CDR-L2, and CDR-L3 of the mouse antibody AbF46 were defined according to Kabat numbering and introduced into the Vk1 subtype. Hereupon, back mutations were conducted at positions 36 (Y→H), 46 (L→M), and 49 (Y→I) on H4-light.

Thereafter, DNA fragments having the heavy chain nucleotide sequences (H1-heavy: SEQ ID NO: 47, H3-heavy: SEQ ID NO: 48, H4-heavy: SEQ ID NO: 49) and DNA fragments having the light chain nucleotide sequences (H1-light: SEQ ID NO: 50, H2-light: SEQ ID NO: 51, H3-light: SEQ ID NO: 52, H4-light: SEQ ID NO: 53) were digested with EcoRI (NEB, R0101S) and XhoI (NEB, R0146S) before cloning into a pOptiVEC™-TOPO TA Cloning Kit enclosed in an OptiCHO™ Antibody Express Kit (Cat no. 12762-019, Invitrogen) and a pcDNA™3.3-TOPO TA Cloning Kit (Cat no. 8300-01), respectively, so as to construct recombinant vectors for expressing a humanized antibody.

Each of the constructed vectors was amplified using Qiagen Maxiprep kit (Cat no. 12662), and a transient expression was performed using Freestyle™ MAX 293 Expression System (invitrogen). 293 F cells were used for the expression and cultured in FreeStyle™ 293 Expression Medium in a suspension culture manner. At one day before the transient expression, the cells were provided in the concentration of 5×10⁵ cells/ml, and after 24 hours, when the cell number reached to 1×10⁶ cells/ml, the transient expression was performed. A transfection was performed by a liposomal reagent method using Freestyle™ MAX reagent (invitrogen), wherein in a 15 ml tube, the DNA was provided in the mixture ratio of 1:1 (heavy chain DNA: light chain DNA) and mixed with 2 ml of OptiPro™ SFM (invtrogen) (tube A), and in another 15 ml tube, 100 ul (microliter) of Freestyle™ MAX reagent and 2 ml of OptiPro™ SFM were mixed (tube B), followed by mixing tube A and tube B and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE healthcare, 17-0405-03), followed by elution with an IgG elution buffer (Thermo Scientific, 21004). The buffer was exchanged with PBS to purify a humanized antibody AbF46 (hereinafter referred to as “huAbF46”). The humanized antibody huAbF46 used in the following examples comprised a combination of H4-heavy (SEQ ID NO: 42) and H4-light (SEQ ID NO: 46).

1.4. Construction of scFV Library of huAbF46 Antibody

For use in constructing an scFv of the huAbF46 antibody from the heavy and light chain variable regions of the huAbF46 antibody, a gene was designed to have the structure of “VH-linker-VL” for each of the heavy and the light chain variable region, with the linker having the amino acid sequence “GLGGLGGGGSGGGGSGGSSGVGS” (SEQ ID NO: 54). A polynucleotide sequence (SEQ ID NO: 55) encoding the designed scFv of huAbF46 was synthesized in Bioneer and an expression vector for the polynucleotide had the nucleotide sequence of SEQ ID NO: 56.

After expression, the product was found to exhibit specificity to c-Met.

1.5. Construction of Library Genes for Affinity Maturation

1.5.1. Selection of Target CDRs and Synthesis of Primers

The affinity maturation of huAbF46 was achieved in the following steps. First, six complementary determining regions (CDRs) were defined according to Kabat numbering. The CDRs are given in Table 1, below.

TABLE 1

| CDR | Amino Acid Sequence |
|---|---|
| CDR-H1 | DYYMS (SEQ ID NO: 1) |
| CDR-H2 | FIRNKANGYTTEYSASVKG (SEQ ID NO: 2) |
| CDR-H3 | DNWFAY (SEQ ID NO: 3) |
| CDR-L1 | KSSQSLLASGNQNNYLA (SEQ ID NO: 10) |
| CDR-L2 | WASTRVS (SEQ ID NO: 11) |
| CDR-L3 | QQSYSAPLT (SEQ ID NO: 12) |

For use in the introduction of random sequences into the CDRs of the antibody, primers were designed as follows. Conventionally, N codons were utilized to introduce bases at the same ratio (25% A, 25% G, 25% C, 25% T) into desired sites of mutation. In this experiment, the introduction of random bases into the CDRs of huAbF46 was conducted in such a manner that, of the three nucleotides per codon in the wild-type polynucleotide encoding each CDR, the first and second nucleotides conserved over 85% of the entire sequence while the other three nucleotides were introduced at the same percentage (each 5%) and that the same possibility was imparted to the third nucleotide (33% G, 33% C, 33% T).

1.5.2. Construction of a Library of huAbF46 Antibodies and Affinity for c-Met

The construction of antibody gene libraries through the introduction of random sequences was carried out using the primers synthesized in the same manner as in Reference Example 1.5.1. Two PCR products were obtained using a polynucleotide covering the scFV of huAbF46 as a template, and were subjected to overlap extension PCR to give scFv library genes for huAbF46 antibodies in which only desired CDRs were mutated. Libraries targeting each of the six CDRs prepared from the scFV library genes were constructed.

The affinity for c-Met of each library was compared to that of the wildtype. Most libraries were lower in affinity for c-Met, compared to the wild-type. The affinity for c-Met was retained in some mutants.

1.6. Selection of Antibody with Improved Affinity from Libraries

After affinity maturation of the constructed libraries for c-Met, the nucleotide sequence of scFv from each clone was analyzed. The nucleotide sequences thus obtained are summarized in Table 2 and were converted into IgG forms. Four antibodies which were respectively produced from clones L3-1, L3-2, L3-3, and L3-5 were used in the subsequent experiments.

TABLE 2

| | Library | |
|---|---|---|
| Clone | constructed | CDR Sequence |
| H11-4 | CDR-H1 | PEYYMS (SEQ ID NO: 22) |
| YC151 | CDR-H1 | PDYYMS (SEQ ID NO: 23) |
| YC193 | CDR-H1 | SDYYMS (SEQ ID NO: 24) |
| YC244 | CDR-H2 | RNNANGNT (SEQ ID NO: 25) |
| YC321 | CDR-H2 | RNKVNGYT (SEQ ID NO: 26) |
| YC354 | CDR-H3 | DNWLSY (SEQ ID NO: 27) |
| YC374 | CDR-H3 | DNWLTY (SEQ ID NO: 28) |
| L1-1 | CDR-L1 | KSSHSLLASGNQNNYLA (SEQ ID NO: 29) |
| L1-3 | CDR-L1 | KSSRSLLSSGNHKNYLA (SEQ ID NO: 30) |
| L1-4 | CDR-L1 | KSSKSLLASGNQNNYLA (SEQ ID NO: 31) |
| L1-12 | CDR-L1 | KSSRSLLASGNQNNYLA (SEQ ID NO: 32) |
| L1-22 | CDR-L1 | KSSHSLLASGNQNNYLA (SEQ ID NO: 33) |
| L2-9 | CDR-L2 | WASKRVS (SEQ ID NO: 34) |
| L2-12 | CDR-L2 | WGSTRVS (SEQ ID NO: 35) |
| L2-16 | CDR-L2 | WGSTRVP (SEQ ID NO: 36) |
| L3-1 | CDR-L3 | QQSYSRPYT (SEQ ID NO: 13) |
| L3-2 | CDR-L3 | GQSYSRPLT (SEQ ID NO: 14) |
| L3-3 | CDR-L3 | AQSYSHPFS (SEQ ID NO: 15) |
| L3-5 | CDR-L3 | QQSYSRPFT (SEQ ID NO: 16) |
| L3-32 | CDR-L3 | QQSYSKPFT (SEQ ID NO: 37) |

1.7. Conversion of Selected Antibodies into IgG

Respective polynucleotides encoding heavy chains of the four selected antibodies were designed to have the structure of “EcoRI-signal sequence-VH-NheI-CH-XhoI” (SEQ ID NO: 38). The heavy chains of huAbF46 antibodies were used as they were because their amino acids were not changed during affinity maturation. In the case of the hinge region, however, the U6-HC7 hinge (SEQ ID NO: 57) was employed instead of the hinge of human IgG1. Genes were also designed to have the structure of “EcoRI-signal sequence-VL-BsiWI-CL-XhoI” for the light chain. Polypeptides encoding light chain variable regions of the four antibodies which were selected after the affinity maturation were synthesized in Bioneer. Then, a DNA fragment having the heavy chain nucleotide sequence (SEQ ID NO: 38) and DNA fragments having the light chain nucleotide sequences (DNA fragment comprising L3-1-derived CDR-L3: SEQ ID NO: 58, DNA fragment comprising L3-2-derived CDR-L3: SEQ ID NO: 59, DNA fragment comprising L3-3-derived CDR-L3: SEQ ID NO: 60, and DNA fragment comprising L3-5-derived CDR-L3: SEQ ID NO: 61) were digested with EcoRI (NEB, R0101S) and XhoI (NEB, R0146S) before cloning into a pOptiVEC™-TOPO TA Cloning Kit enclosed in an OptiCHO™ Antibody Express Kit (Cat no. 12762-019, Invitrogen) and a pcDNA™3.3-TOPO TA Cloning Kit (Cat no. 8300-01), respectively, so as to construct recombinant vectors for expressing affinity-matured antibodies.

Each of the constructed vectors was amplified using Qiagen Maxiprep kit (Cat no. 12662), and a transient expression was performed using Freestyle™ MAX 293 Expression System (invitrogen). 293 F cells were used for the expression and cultured in FreeStyle™ 293 Expression Medium in a suspension culture manner. At one day before the transient expression, the cells were provided in the concentration of 5×10⁵ cells/ml, and after 24 hours, when the cell number reached to 1×10⁶ cells/ml, the transient expression was performed. A transfection was performed by a liposomal reagent method using Freestyle™ MAX reagent (invitrogen), wherein in a 15 ml tube, the DNA was provided in the mixture ratio of 1:1 (heavy chain DNA: light chain DNA) and mixed with 2 ml of OptiPro™ SFM (invtrogen) (tube A), and in another 15 ml tube, 100 ul (microliter) of Freestyle™ MAX reagent and 2 ml of OptiPro™ SFM were mixed (tube B), followed by mixing tube A and tube B and incubating for 15 minutes. The obtained mixture was slowly mixed with the cells provided one day before the transient expression. After completing the transfection, the cells were incubated in 130 rpm incubator for 5 days under the conditions of 37 t, 80% humidity, and 8% CO₂.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE healthcare, 17-0405-03), followed by elution with an IgG elution buffer (Thermo Scientific, 21004). The buffer was exchanged with PBS to purify four affinity-matured antibodies (hereinafter referred to as “huAbF46-H4-A1 (L3-1 origin), huAbF46-H4-A2 (L3-2 origin), huAbF46-H4-A3 (L3-3 origin), and huAbF46-H4-A5 (L3-5 origin),” respectively).

1.8. Construction of Constant Region- and/or Hinge Region-Substituted huAbF46-H4-A1

Among the four antibodies selected in Reference Example 1.7, huAbF46-H4-A1 was found to be the highest in affinity for c-Met and the lowest in Akt phosphorylation and c-Met degradation degree. In the antibody, the hinge region, or the constant region and the hinge region, were substituted.

The antibody huAbF46-H4-A1 (U6-HC7) was composed of a heavy chain comprising the heavy chain variable region of huAbF46-H4-A1, U6-HC7 hinge, and the constant region of human IgG1 constant region, and a light chain comprising the light chain variable region of huAbF46-H4-A1 and human kappa constant region. The antibody huAbF46-H4-A1 (IgG2 hinge) was composed of a heavy chain comprising a heavy chain variable region, a human IgG2 hinge region, and a human IgG1 constant region, and a light chain comprising the light chain variable region of huAbF46-H4-A1 and a human kappa constant region. The antibody huAbF46-H4-A1 (IgG2 Fc) was composed of the heavy chain variable region of huAbF46-H4-A1, a human IgG2 hinge region, and a human IgG2 constant region, and a light chain comprising the light variable region of huAbF46-H4-A1 and a human kappa constant region. Hereupon, the histidine residue at position 36 on the human kappa constant region of the light chain was changed to tyrosine in all of the three antibodies to increase antibody production.

For use in constructing the three antibodies, a polynucleotide (SEQ ID NO: 63) encoding a polypeptide (SEQ ID NO: 62) composed of the heavy chain variable region of huAbF46-H4-A1, a U6-HC7 hinge region, and a human IgG1 constant region, a polynucleotide (SEQ ID NO: 65) encoding a polypeptide (SEQ ID NO: 64) composed of the heavy chain variable region of huAbF46-H4-A1, a human IgG2 hinge region, and a human IgG1 region, a polynucleotide (SEQ ID NO: 67) encoding a polypeptide (SEQ ID NO: 66) composed of the heavy chain variable region of huAbF46-H4-A1, a human IgG2 region, and a human IgG2 constant region, and a polynucleotide (SEQ ID NO: 69) encoding a polypeptide (SEQ ID NO: 68) composed of the light chain variable region of huAbF46-H4-A1, with a tyrosine residue instead of histidine at position 36, and a human kappa constant region were synthesized in Bioneer. Then, the DNA fragments having heavy chain nucleotide sequences were inserted into a pOptiVEC™-TOPO TA Cloning Kit enclosed in an OptiCHO™ Antibody Express Kit (Cat no. 12762-019, Invitrogen) while DNA fragments having light chain nucleotide sequences were inserted into a pcDNA™3.3-TOPO TA Cloning Kit (Cat no. 8300-01) so as to construct vectors for expressing the antibodies.

Each of the constructed vectors was amplified using Qiagen Maxiprep kit (Cat no. 12662), and a transient expression was performed using Freestyle™ MAX 293 Expression System (invitrogen). 293 F cells were used for the expression and cultured in FreeStyle™ 293 Expression Medium in a suspension culture manner. At one day before the transient expression, the cells were provided in the concentration of 5×10⁵ cells/ml, and after 24 hours, when the cell number reached to 1×10⁶ cells/ml, the transient expression was performed. A transfection was performed by a liposomal reagent method using Freestyle™ MAX reagent (invitrogen), wherein in a 15 ml tube, the DNA was provided in the mixture ratio of 1:1 (heavy chain DNA: light chain DNA) and mixed with 2 ml of OptiPro™ SFM (invtrogen) (tube A), and in another 15 ml tube, 100 ul (microliter) of Freestyle™ MAX reagent and 2 ml of OptiPro™ SFM were mixed (tube B), followed by mixing tube A and tube B and incubating for 15 minutes. The obtained mixture was slowly mixed with the cells provided one day before the transient expression. After completing the transfection, the cells were incubated in 130 rpm incubator for 5 days under the conditions of 37° C., 80% humidity, and 8% CO₂.

After centrifugation, the supernatant was applied to AKTA prime (GE Healthcare) to purify the antibody. In this regard, 100 mL of the supernatant was loaded at a flow rate of 5 mL/min to AKTA Prime equipped with a Protein A column (GE healthcare, 17-0405-03), followed by elution with IgG elution buffer (Thermo Scientific, 21004). The buffer was exchanged with PBS to finally purify three antibodies (huAbF46-H4-A1 (U6-HC7), huAbF46-H4-A1 (IgG2 hinge), and huAbF46-H4-A1 (IgG2 Fc)). Among the three antibodies, huAbF46-H4-A1 (IgG2 Fc) was representatively selected for the following examples, and referred as anti-c-Met antibody L3-1Y/IgG2.

### Example 1

### Decreased Interaction Between LRIG1 and USP8 by Anti-c-Met Antibody

LRIG1 (AAU44786) was overexpressed in MKN45 gastric cancer cells (JCRB, JCRB0254) using lipofectamin reagent (Invitrogen). Forty-eight hours after, the obtained LRIG1-overexpressed MKN45 gastric cancer cells were treated with 5 ug/ml of anti-c-Met antibody L3-1Y/IgG2 for 1 hour, and the interaction between LRIG1 (AAU44786) and USP8 (NP_{—}001122082: SEQ ID NO: 109) was measured. For this experiment, MKN45 gastric cancer cells were cultured in RPMI media containing 10% (v/v) FBS, and 10 ml of the cells (2×10⁵ cells/ml) were inoculated on 100 mm dish. To the 100 mm dish, the mixture of 10 ug of Flag-LRIG1 DNA and 40 ul of lipofectamin reagent (Invitrogen) was added to perform the transfection of the cells, and 48 hours after, 5 ug/ml of anti-c-Met antibody L3-1Y/IgG2 was treated.

An immunoprecipitation was conducted with the L3-1Y/IgG2 antibody treated cells using anti-Flag antibody (Sigma). The cell culture (4 mL) was treated with 100 uM of concanamycin, and 4 hours after, the cells were collected. The collected cells were lysed with lysis buffer (Complete Lysis-M, Roche) and centrifuged at 13000 rpm for 15 minutes at 4° C. to obtain a protein solution. An immunoblotting was conducted for the obtained protein solution using anti-LRIG1 antibody (Abcam, Cambridge, UK) or anti-USP8 antibody (Cell Signaling, Danvers, Mass., USA).

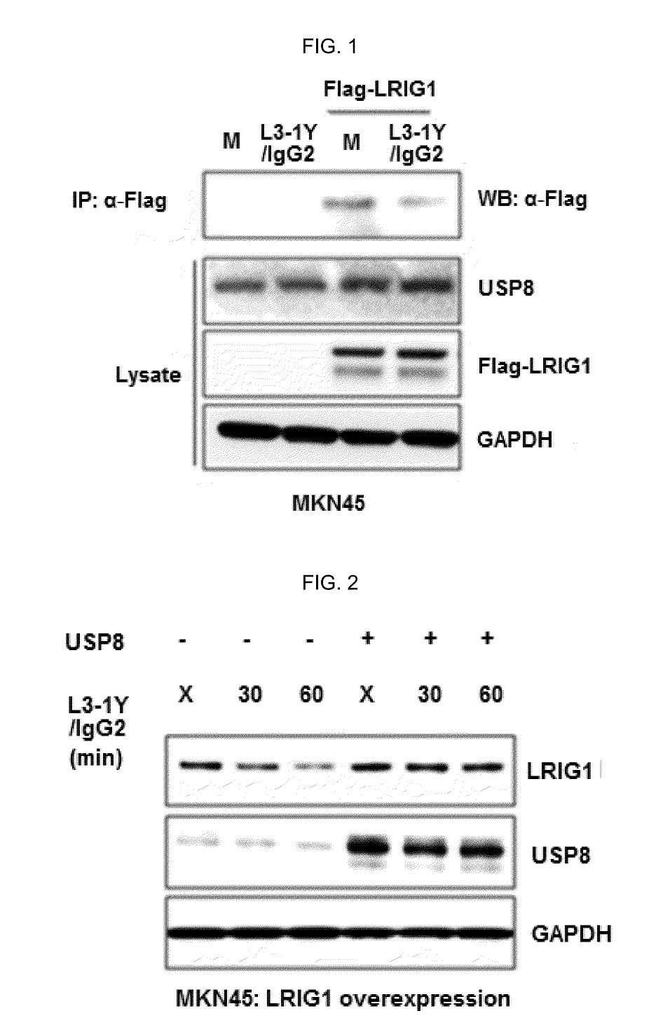

The obtained results are shown in FIG. 1. As shown in FIG. 1, the level of USP8 interacting with LRIG1 was decreased by treatment of anti-c-Met antibody L3-1Y/IgG2.

### Example 2

### Inhibition of USP8 Against Degradation of LRIG1 by Anti-c-Met Antibody

Increased USP8 levels lead to increased stability of LRIG1. It was confirmed that when LRIG1-overexpressed MKN45 cells were treated with anti-c-Met antibody (5 ug/ml), the degradation of LRIG1 was stimulated, whereas in USP8-overexpressed MKN45 cells, LRIG1 was not degraded by anti-c-Met antibody.

In particular, LRIG1 (AAU44786) and USP8 (NP_{—}001122082) were overexpressed in MKN45 gastric cancer cells (JCRB, JCRB0254) using lipofectamin reagent. Forty-eight hours after, the cells were treated with L3-1Y/IgG2 (5 ug/ml), and cultured for the time indicated in FIG. 2 (30 minutes or 60 minutes). The MKN45 gastric cancer cells were cultured in 10% FBS RPMI media, and in 100 mm dish, the cells were transfected using the mixture of 5 ug of Flag-LRIG1 DNA, 5 ug or Flag-USP8 DNA and 40 ul lipofectamin reagent. Forty-eight hours after the transfection, the transfected cells were treated with 5 ug/ml of L3-1Y/IgG2 antibody.

An immunoprecipitation was conducted for the L3-1Y/IgG2 antibody treated cells using anti-Flag antibody (Sigma). The cells were treated with 100 uM concanamycin. 4 hours after, the cells were collected and lysed with lysis buffer. Then, the cell lysate was centrifuged at 13000 rpm for 15 minutes at 4° C. to obtain protein solution. For the protein solution, an immunoblotting was conducted using anti-LRIG1 antibody (Abcam, Cambridge, UK) or anti-USP8 antibody (Cell Signaling, Danvers, Mass., USA).

The obtained results are shown in FIG. 2. As shown in FIG. 2, LRIG1 was degraded by the treatment of anti-c-Met antibody L3-1Y/IgG2, and the degree of LRIG1 degradation was decreased by the present of USP8.

### Example 3

### Examination on Relation Between Expression Level of USP8 and Ubiquitination of LRIG1 by Anti-c-Met Antibody

To confirm the deubiquitination of LRIG1 by USP8, it was tested whether the ubiquitination of LRIG1 by anti-c-Met antibody is decreased in USP8-overexpressed EBC1 cells.

In particular, Flag-USP8 was overexpressed in EBC1 cells (JCRB JCRB0820) using lipofectamin reagent. Forty-eight hours after, the cells were treated with 5 ug/ml of anti-c-Met antibody L3-1Y/IgG2 for 1 hour. EBC1 cells (JCRB JCRB0820) were cultured in 10% FBS RPMI media, and in 100 mm dish, the cells were transfected using the mixture of 10 ug of Flag-USP8 DNA and 40 ul of lipofectamin reagent. Forty-eight hours after transfection, the transfected cells were treated with 5 ug/ml of the antibody.

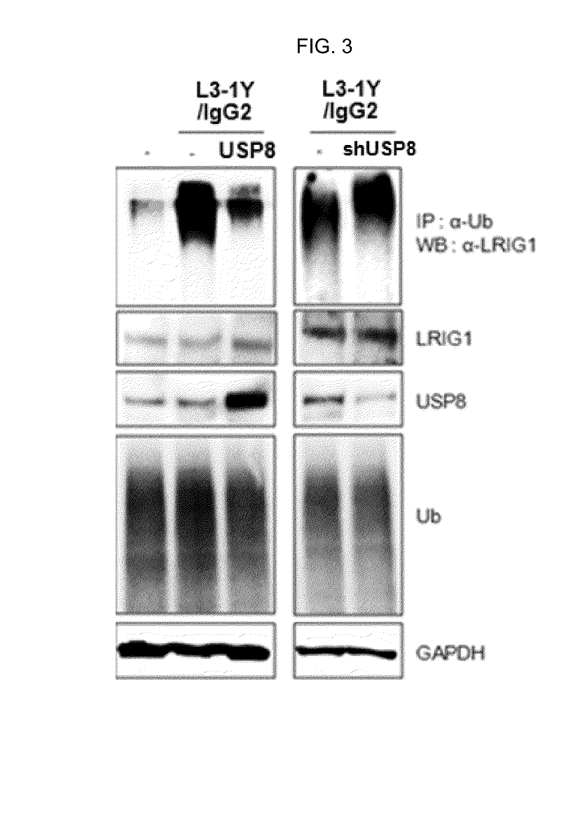

An immunoprecipitation was conducted for the L3-1Y/IgG2 antibody treated cells using anti-Ubiquitin antibody (Santa Cruz). The cells were treated with 100 uM concanamycin. 4 hours after, the cells were collected and lysed with lysis buffer. Then, the cell lysate was centrifuged at 13000 rpm for 15 minutes at 4° C. to obtain protein solution. For the protein solution, an immunoprecipitation was conducted using anti-Ubiquitin antibody (Santa Cruz), and then, an immunoblotting was conducted using anti-LRIG1 antibody (Abcam, Cambridge, UK) or anti-Ubiquitin antibody. The obtained results are shown in FIG. 3 (left).

Meanwhile, to confirm that the ubiquitination of LRIG1 is stimulated by inhibition of USP8, it was examined whether the ubiquitination of LRIG1 is increased in USP8 knock-down EBC1 cells. In particular, EBC1 cells were transfected with control vector (Dharmacon) or shUSP8 (SEQ ID NO: 112; shUSP8 mature antisense: tatctcttccgattatcag) mixed with 10 ug of DNA and 40 ul of lipofectamin reagent. The transfected cells were treated with 5 ug/ml of L3-1Y/IgG2 and cultured at 37° C. for 1 hour. The obtained results are shown in FIG. 3 (right).

In FIG. 3, on the left are the results obtained by overexpression of wild type USP8, and on the right are the results obtained by knock-down of USP8. As shown in FIG. 3, the degree of ubiquitination of LRIG1 by the treatment of L3-1Y/IgG2 IgG2 is varied depending on the expression level of USP8, that is, whether or not the USP8 gene is knocked down or not.

### Example 4

### Examination on Anticancer Efficacy of Anti-c-Met Antibody by Knock-Down of USP8

Seventy-two hours after knock down of USP8 in EBC1 cells, the level of cell proliferation was measured by CTG assay.

In particular, in 96 well plate, a reverse transfection of EBC1 cells (JCRB JCRB0820) using shUSP8(SEQ ID NO: 112) was performed to knock down the cells, referring to the method of Example 3. 24 hours after, the USP8 knock-down cells were treated with L3-1Y/IgG2 in the amount of Oug/ml, 0.016 ug/ml, 0.08 ug/ml, 0.4 ug/ml, or 2 ug/ml for 72 hours. To the 96 well plate where the cells were cultured, Cell Titer Glo solution (Promega) was added in the amount of 100 ul per well, and 30 minutes after, luminescence signal was measured with Envision 2104 Multi-label Reader (Perkin Elmer, Foster City, Calif., USA).

For comparison, the same experiment was performed using the control vector reverse-transfected cells.

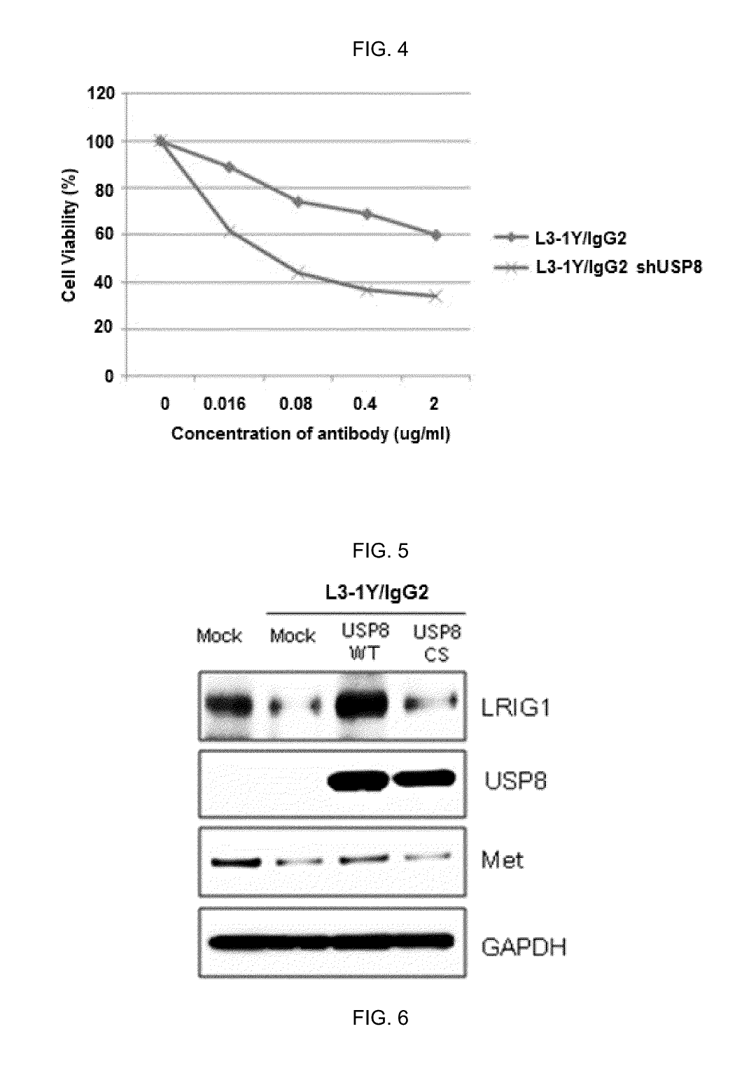

The obtained cell viability (%) is shown in FIG. 4. As shown in FIG. 4, the anticancer efficacy of L3-1Y/IgG2 IgG2 on the cells with USP8 knock down is considerably increased compared with that on the cells without USP8 knock down.

### Example 5

### Examination of the Change in c-Met Degradation Activity by USP8 Mutation

In this example, USP8 wild type and USP8 C786S mutant were respectively overexpressed in EBC1 cells. It was confirmed that when the cells were treated with anti-c-Met antibody (5 ug/ml), degradation of LRIG1 is stimulated, where LRIG1 degradation by anti-c-Met antibody is induced in USP8 C786S mutant overexpressed cells, but not induced in USP8 wild type overexpressed cell. These results indicated that the increased level of USP8 leads to increase of LRIG1 stability (decrease of LRIG1 degradation), whereas the mutation in the active domain of USP8 leads to decrease of LRIG1 stability.

In particular, to examine whether or not USP8 affects the c-Met degradation activity of anti-c-Met antibody, USP8-WT (SEQ ID NO: 110) or inactive USP8-CS mutant (coding gene for SEQ ID NO: 111) were overexpressed in EBC1 cells (JCRB JCRB0820) using lipofectamin reagent. Forty-eight hours after, the cells were treated with L3-1Y/IgG2 (5 ug/ml) for 1 hour. The EBC1 cells (JCRB JCRB0820) were cultured in 10% FBS RPMI media, and in 100 mm dish, the cells were transfected by 10 ug of mixing Flag-LRIG1 DNA 10 ug and 40 ul lipofectamin reagent, and 48 hours after, treated with L3-1Y/IgG2 (5 ug/ml).

The L3-1Y/IgG2 antibody treated cells were collected, lysed with lysis buffer, and then centrifuged at 13000 rpm for 15 minutes at 4° C. to obtain protein solution. For the obtained protein solution, an immunoblotting was conducted using anti-c-Met antibody (Santa cruz, biotechnology Inc) or anti-LRIG1 antibody (Abcam, Cambridge, UK).

The obtained results are shown in FIG. 5. As shown in FIG. 5, when wild type USP8 is overexpressed, the c-Met degradation by L3-1Y/IgG2 is inhibited, whereas when inactive USP8 mutant was overexpressed, the c-Met degradation by L3-1Y/IgG2 is increased. The results indicate that the c-Met degradation activity of L3-1Y/IgG2 differs depending on the activation or inactivation of USP8.

### Example 6

### Screening of Anti-c-Met Antibody Sensitive Cancer Cells Using USP8

To confirm the role of USP8 in tumor suppression, the following experiment was performed. Referring to the method of Example 5, EBC1 cells (JCRB JCRB0820) were transfected with USP8-WT (SEQ ID NO: 110), inactive USP8-CS mutant (coding gene for SEQ ID NO: 111), or shUSP8 (SEQ ID NO: 112) containing plasmid, to overexpress the genes.

Using Celltiter Glo (CTG) luminescent assay, the degree of cell growth of the EBC1 cells by treatment of anti-c-Met antibody (in vitro experimentation) was examined. The EBC1 cells were inoculated on 96-well plate (BD Biosciences, Palo Alto, Calif., USA) in the density of 5×10³ cells per each well, and cultured in FBS 10% (v/v) RPMI 1640 medium (Gibco). 24 hours after, L3-1Y/IgG2 antibody was treated in the amount of 2 ug/ml which was diluted with 100 uL of 10% FBS (v/v) RPMI medium. 72 hours after, 100 uL of CTG solution (Promega) was added to each well, and the cells in the well were further cultured at 37° C. for 30 minutes. The luminescence signal from the cell culture was detected and recorded using Envision 2104 Multi-label Reader (Perkin Elmer). The cells transfected using control empty vector were used as a control (CTL).

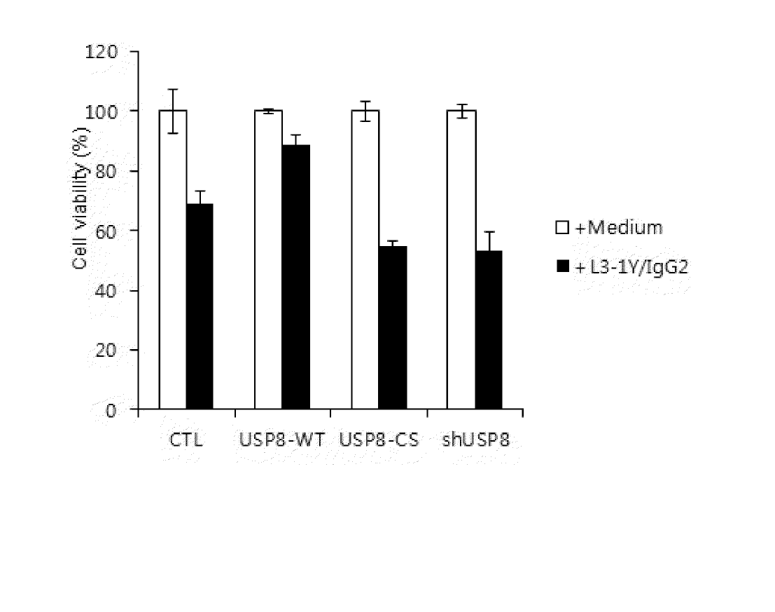

The obtained results are shown in FIG. 6. As shown in FIG. 6, the inhibition against the growth of EBC1 cells by L3-1Y/IgG2 is not achieved by the overexpression of wild-type USP8, but can be achieved by the overexpression of USP8-CS or shUSP8.

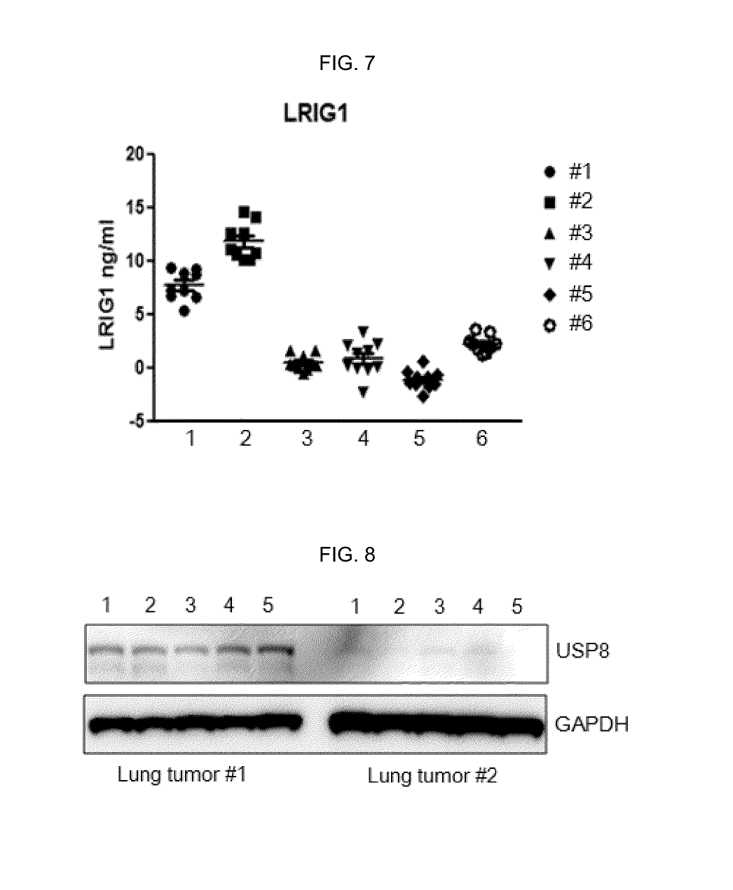

Based on the results, in vivo anti-tumor effect of L3-1Y/IgG2 antibody were determined using patient derived lung tumor xenograft samples (prepared by Oncotest, where cancer cells derived from lung cancer patients were grafted into male NRMI nu/nu mice). Based on the level of Met mRNA, 6 xenograft samples (#1, #2, #3, #4, #5, and #6) of lung tumor were selected, and the level of LRIG1 protein was measured using LRIG1 ELISA kit (MBS908302, MyBioSoure, CA, USA). The obtained results are shown in FIG. 7 (horizontal axis: samples, vertical axis: concentration of LRIG1).

Based on the results of FIG. 7, lung tumor xenograft samples #1 and #2 having high level of LRIG1 protein were selected, and the USP8 levels in the biological samples were measured by western blotting using anti-USP8 antibody (Cell Signaling, Danvers, Mass., USA). The obtained results are shown in FIG. 8. As shown in FIG. 8, in lung tumor xenograft sample #1, the USP8 level is relatively high whereas in lung tumor xenograft sample #2, USP8 is nearly absent or present in low level.

Through the above step, lung tumor xenograft samples #1 and #2 which have high level of LRIG1 but different level of USP8 from each other. Lung tumor xenograft samples #1 and #2 were treated with L3-1Y/IgG2 antibody (5 mg/kg I.V. once/a week) and then ground to obtain proteins. The level of c-Met protein in the obtained proteins was measured using total c-Met ELISA kit (R&D systems). PBS treated group (group without antibody treatment) was used as a control.

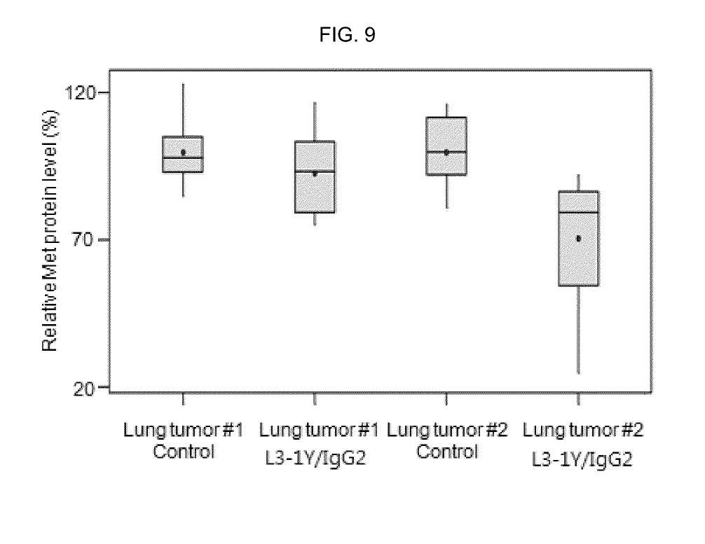

The obtained results are shown in FIG. 9. The c-Met level in the antibody treated sample is indicated as a percentage (%) of the c-Met level in the control. As shown in FIG. 9, in the xenograft sample #2 with low level of USP8, the c-Met level is decreased by treating with L3-1Y/IgG2 antibody, whereas in the xenograft sample #1 with high level of USP8, the c-Met level is not decreased by treating with L3-1Y/IgG2 antibody.

To confirm in vivo effect of the anti-c-Met antibody on the growth of patient derived lung tumor cells, tumor xenografting test was performed using male NRMI nu/nu mice (performed by Oncotest). The mice were acclimated for at least 1 week before tumor inoculation. Then, the mice were anesthetized with 1-2% (v/v) isofuran, and patient derived lung tumor cells (5×10⁶ cells) were injected to right flank of the mice. 7 days after, when the tumor cells grow to the average size of 50-250 mm³, the mice were grouped into follow groups: antibody L3-1Y/IgG2 (5 mg/kg I.V. once/a week) treated group, and vehicle (PBS 0.2 ml I.V. once/a week) treated group (control). Each group consisted of 10 mice. The tumor size and weight of mice were measured 2-3 times per a week during the experimentation period of 6 weeks in total. The tumor size (V) was calculated as follows: V(mm³)={long axis length (mm)×(short axis length (mm))²}/2.

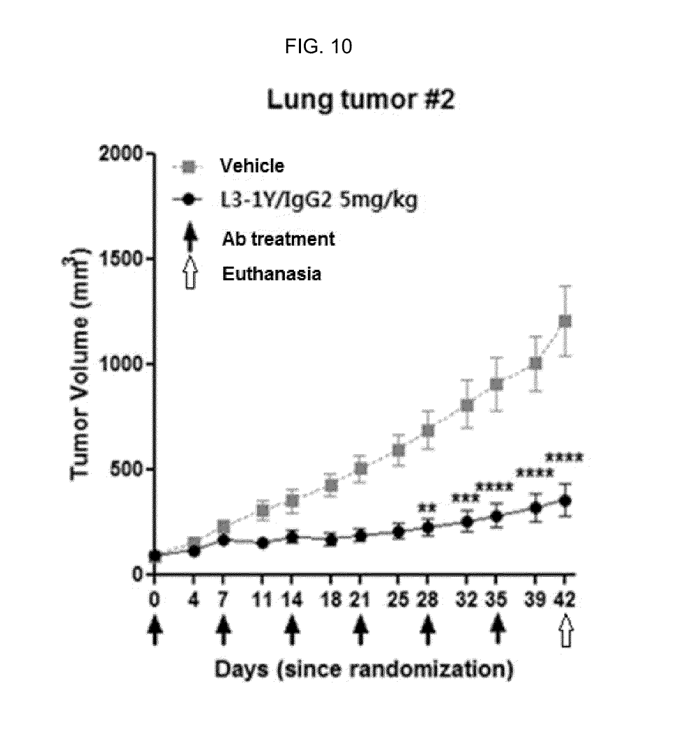

The obtained results are shown in FIG. 10. As shown in FIG. 10, similar to the results of decrease of c-Met, the inhibition effect by treatment of L3-1Y/IgG2 antibody is observed in xenograft sample #2 (about 76% decrease).

From the above results, it can be revealed that L3-1Y/IgG2 antibody exhibits more excellent c-Met degradation effect in tumor having low level of USP8.

All references, including publications, patent applications, and patents, cited herein are hereby incorporated by reference to the same extent as if each reference were individually and specifically indicated to be incorporated by reference and were set forth in its entirety herein.
The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.
Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of selecting a subject for administration of an anti-c-Met antibody, comprising
(i) determining the presence or the amount of a ubiquitin peptidase or a ubiquitin peptidase-coding gene, or measuring the activity of a ubiquitin peptidase, in a biological sample from a subject; and
(ii) selecting the subject for administration of an anti-c-Met antibody if the amount of ubiquitin peptidase or ubiquitin peptidase-coding gene, or activity level of ubiquitin peptidase, in the sample is absent or at a low level as compared to a reference sample in which the anti-c-Met antibody has no effect or has a resistance to the anti-c-Met antibody, or the activity level has a score measured by immunohistochemical staining of “−”, “0”, or “+1.”

2. The method according to claim 1, wherein the ubiquitin peptidase is ubiquitin specific peptidase 8 (USPS).

3. The method according to claim 1, wherein the anti-c-Met antibody is an antibody or an antigen-binding fragment thereof comprising:
at least one heavy chain complementarity determining region (CDR) selected from the group consisting of (a) a CDR-H1 comprising the amino acid sequence of SEQ ID NO: 4; (b) a CDR-H2 comprising the amino acid sequence of SEQ ID NO: 5, SEQ ID NO: 2, or an amino acid sequence comprising 8-19 consecutive amino acids of SEQ ID NO: 2 including the 3^{rd} to 10^{th} positions of SEQ ID NO: 2; and (c) a CDR-H3 comprising the amino acid sequence of SEQ ID NO: 6, SEQ ID NO: 85, or an amino acid sequence comprising 6-13 consecutive amino acids of SEQ ID NO: 85 including the 1^{st} to 6^{th} positions of SEQ ID NO: 85, or a heavy chain variable region comprising the at least one heavy chain complementarity determining region;
at least one light chain complementarity determining region selected from the group consisting of (a) a CDR-L1 comprising the amino acid sequence of SEQ ID NO: 7, (b) a CDR-L2 comprising the amino acid sequence of SEQ ID NO: 8, and (c) a CDR-L3 comprising the amino acid sequence of SEQ ID NO: 9, SEQ ID NO: 86, or an amino acid sequence comprising 9-17 consecutive amino acids of SEQ ID NO: 89 including the 1^{st} to 9^{th} positions of SEQ ID NO: 89, or a light chain variable region comprising the at least one light chain complementarity determining region;
a combination of the at least one heavy chain complementarity determining region and the at least one light chain complementarity determining region; or
a combination of the heavy chain variable region and the light chain variable region.

4. A method of preventing or treating cancer comprising co-administering (a) an anti-c-Met antibody or an antigen-bonding fragment thereof and (b) an inhibitor of a ubiquitin peptidase or a ubiquitin peptidase coding gene to a subject in need of prevention or treatment of cancer.

5. The method according to claim 4, wherein the anti-c-Met antibody or the antigen-bonding fragment thereof and the inhibitor against the ubiquitin peptidase or the ubiquitin peptidase coding gene are co-administered simultaneously or sequentially in any order.

6. The method according to claim 4, wherein the ubiquitin peptidase is ubiquitin specific peptidase 8 (USPS).

7. The method according to claim 4, wherein the inhibitor against the ubiquitin peptidase or the ubiquitin peptidase coding gene comprises at least one selected from the group consisting of:
a mutant ubiquitin peptidase comprising a mutation in an active site, a chemical inhibitor of the ubiquitin peptidase, an antibody against the ubiquitin peptidase, and an aptamer against the ubiquitin peptidase; and a chemical inhibitor or an aptamer against the ubiquitin peptidase coding gene, an siRNA against the ubiquitin peptidase coding gene, a microRNA against the ubiquitin peptidase coding gene, an shRNA against the ubiquitin peptidase coding gene, and a polynucleotide encoding a mutant ubiquitin peptidase comprising a mutation in an active site.

8. The method according to claim 4, wherein the anti-c-Met antibody or the antigen-binding fragment thereof comprises:
at least one heavy chain complementarity determining region (CDR) selected from the group consisting of (a) a CDR-H1 comprising the amino acid sequence of SEQ ID NO: 4; (b) a CDR-H2 comprising the amino acid sequence of SEQ ID NO: 5, SEQ ID NO: 2, or an amino acid sequence comprising 8-19 consecutive amino acids of SEQ ID NO: 2 including the 3^{rd} to 10^{th} positions of SEQ ID NO: 2; and (c) a CDR-H3 comprising the amino acid sequence of SEQ ID NO: 6, SEQ ID NO: 85, or an amino acid sequence comprising 6-13 consecutive amino acids of SEQ ID NO: 85 including the 1^{st} to 6^{th} positions of SEQ ID NO: 85, or a heavy chain variable region comprising the at least one heavy chain complementarity determining region;
at least one light chain complementarity determining region selected from the group consisting of (a) a CDR-L1 comprising the amino acid sequence of SEQ ID NO: 7, (b) a CDR-L2 comprising the amino acid sequence of SEQ ID NO: 8, and (c) a CDR-L3 comprising the amino acid sequence of SEQ ID NO: 9, SEQ ID NO: 86, or an amino acid sequence comprising 9-17 consecutive amino acids of SEQ ID NO: 89 including the 1^{st} to 9^{th} positions of SEQ ID NO: 89, or a light chain variable region comprising the at least one light chain complementarity determining region;
a combination of the at least one heavy chain complementarity determining region and the at least one light chain complementarity determining region; or
a combination of the heavy chain variable region and the light chain variable region.

9. The method according to claim 8, wherein
the CDR-H1 comprises the amino acid sequence of SEQ ID NO: 1, SEQ ID NO: 22, SEQ ID NO: 23, or SEQ ID NO: 24,
the CDR-H2 comprises the amino acid sequence of SEQ ID NO: 2, SEQ ID NO: 25, or SEQ ID NO: 26,
the CDR-H3 comprises the amino acid sequence of SEQ ID NO: 3, SEQ ID NO: 27, SEQ ID NO: 28, or SEQ ID NO: 85,
the CDR-L1 comprises the amino acid sequence of SEQ ID NO: 10, SEQ ID NO: 29, SEQ ID NO: 30, SEQ ID NO: 31, SEQ ID NO: 32, SEQ ID NO: 33, or SEQ ID NO: 106,
the CDR-L2 comprises the amino acid sequence of SEQ ID NO: 11, SEQ ID NO: 34, SEQ ID NO: 35, or SEQ ID NO: 36, and
the CDR-L3 comprises the amino acid sequence of SEQ ID NO: 12, SEQ ID NO: 13, SEQ ID NO: 14, SEQ ID NO: 15, SEQ ID NO: 16, SEQ ID NO: 37, SEQ ID NO: 86, or SEQ ID NO: 89.

10. The method according to claim 8, wherein
the heavy chain variable region comprises an amino acid sequence selected from the group consisting of SEQ ID NOS: 17, 74, 87, 90, 91, 92, 93, and 94, and
the light chain variable region comprises an amino acid sequence selected from the group consisting of SEQ ID NOS: 18, 19, 20, 21, 75, 88, 95, 96, 97, 98, 99, and 107.

11. The method according to claim 8, wherein the anti-c-Met antibody comprises:
(i) a heavy chain comprising the amino acid sequence of SEQ ID NO: 62, the amino acid sequence from the 18^{th} to 462^{nd} positions of SEQ ID NO: 62, the amino acid sequence of SEQ ID NO: 64, the amino acid sequence from the 18^{th} to 461^{st} positions of SEQ ID NO: 64, the amino acid sequence of SEQ ID NO: 66, or the amino acid sequence from the 18^{th} to 460^{th} positions of SEQ ID NO: 66; and
(ii) a light chain comprising the amino acid sequence of SEQ ID NO: 68, the amino acid sequence from the 21^{st} to 240^{th} positions of SEQ ID NO: 68, the amino acid sequence of SEQ ID NO: 70, the amino acid sequence from the 21^{st} to 240^{th} positions of SEQ ID NO: 70, or the amino acid sequence of SEQ ID NO: 108.

12. A method for screening to identify a drug useful for preventing or treating a cancer, comprising:
(i) contacting a candidate compound to a biological sample;
(ii) measuring a level of a ubiquitin peptidase or a ubiquitin peptidase coding gene in the biological sample; and
(iii) comparing the level of the ubiquitin peptidase or the ubiquitin peptidase coding gene in the biological sample contacted by the candidate compound to the level of the ubiquitin peptidase or the ubiquitin peptidase coding gene in a biological sample not contacted by the candidate compound.

13. The method according to claim 12, the drug is a drug to be co-administered with an anti-c-Met antibody.

14. A method of enhancing the efficacy of an anti-c-Met antibody in preventing and/or treating cancer, comprising administering an anti-c-Met antibody to a subject in need thereof and inhibiting a ubiquitin peptidase or a gene for a ubiquitin peptidase in the subject.

15. A method of treating or preventing cancer in a subject comprising administering an anti-c-Met antibody to the subject, wherein the subject has a low level of ubiquitin peptidase expression or activity as compared to the level in a reference sample on which the anti-c-Met antibody has no effect or having a resistance to the anti-c-Met antibody.


SEQUENCE LISTING (112 sequences)

SEQ ID NO: 1 - protein, 5 aa, Artificial Sequence
  Synthetic (heavy chain CDR1 of AbF46)
DYYMS

SEQ ID NO: 2 - protein, 19 aa, Artificial Sequence
  Synthetic (heavy chain CDR2 of AbF46)
FIRNKANGYTTEYSASVKG

SEQ ID NO: 3 - protein, 6 aa, Artificial Sequence
  Synthetic (heavy chain CDR3 of AbF46)
DNWFAY

SEQ ID NO: 4 - protein, 6 aa, Artificial Sequence
  Synthetic (heavy chain CDR1 of c-Met antibody)
XXYYMS

SEQ ID NO: 5 - protein, 8 aa, Artificial Sequence
  Synthetic (heavy chain CDR2 of c-Met antibody)
RNXXNGXT

SEQ ID NO: 6 - protein, 6 aa, Artificial Sequence
  Synthetic (heavy chain CDR3 of c-Met antibody)
DNWLXY

SEQ ID NO: 7 - protein, 17 aa, Artificial Sequence
  Synthetic (light chain CDR1 of c-Met antibody)
KSSXSLLAXGNXXNYLA

SEQ ID NO: 8 - protein, 7 aa, Artificial Sequence
  Synthetic (light chain CDR2 of c-Met antibody)
WXSXRVX

SEQ ID NO: 9 - protein, 9 aa, Artificial Sequence
  Synthetic (light chain CDR3 of c-Met antibody)
XQSYSXPXT

SEQ ID NO: 10 - protein, 17 aa, Artificial Sequence
  Synthetic (light chain CDR1 of AbF46)
KSSQSLLASGNQNNYLA

SEQ ID NO: 11 - protein, 7 aa, Artificial Sequence
  Synthetic (light chain CDR2 of AbF46)
WASTRVS

SEQ ID NO: 12 - protein, 9 aa, Artificial Sequence
  Synthetic (light chain CDR3 of AbF46)
QQSYSAPLT

SEQ ID NO: 13 - protein, 9 aa, Artificial Sequence
  Synthetic (CDR-L3 derived from L3-1 clone)
QQSYSRPYT

SEQ ID NO: 14 - protein, 9 aa, Artificial Sequence
  Synthetic (CDR-L3 derived from L3-2 clone)
GQSYSRPLT

SEQ ID NO: 15 - protein, 9 aa, Artificial Sequence
  Synthetic (CDR-L3 derived from L3-3 clone)
AQSYSHPFS

SEQ ID NO: 16 - protein, 9 aa, Artificial Sequence
  Synthetic (CDR-L3 derived from L3-5 clone)
QQSYSRPFT

SEQ ID NO: 17 - protein, 117 aa, Artificial Sequence
  Synthetic (heavy chain variable region of anti c-Met humanized antibody(huAbF46-H4))
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSS

SEQ ID NO: 18 - protein, 114 aa, Artificial Sequence
  Synthetic (light chain variable region of anti c-Met humanized antibody(huAbF46-H4))
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKR

SEQ ID NO: 19 - protein, 114 aa, Artificial Sequence
  Synthetic (light chain variable region of anti c-Met humanized antibody(huAbF46-H4))
DIQMTQSPSSLSASVGDRVTITCKSSQSLLASGNQNNYLAWHQQKPGKAPKMLIIWASTRVSGVPSRFSGSGSGTDFTLTISSLQPEDFATYYCGQSYSRPLTFGQGTKVEIKR

SEQ ID NO: 20 - protein, 114 aa, Artificial Sequence
  Synthetic (light chain variable region of anti c-Met humanized antibody(huAbF46-H4))
DIQMTQSPSSLSASVGDRVTITCKSSQSLLASGNQNNYLAWHQQKPGKAPKMLIIWASTRVSGVPSRFSGSGSGTDFTLTISSLQPEDFATYYCAQSYSHPFSFGQGTKVEIKR

SEQ ID NO: 21 - protein, 114 aa, Artificial Sequence
  Synthetic (light chain variable region of anti c-Met humanized antibody(huAbF46-H4))
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPFTFGQGTKVEIKR

SEQ ID NO: 22 - protein, 6 aa, Artificial Sequence
  Synthetic (CDR-H1 derived from H11-4 clone)
PEYYMS

SEQ ID NO: 23 - protein, 6 aa, Artificial Sequence
  Synthetic (CDR-H1 derived from YC151 clone)
PDYYMS

SEQ ID NO: 24 - protein, 6 aa, Artificial Sequence
  Synthetic (CDR-H1 derived from YC193 clone)
SDYYMS

SEQ ID NO: 25 - protein, 8 aa, Artificial Sequence
  Synthetic (CDR-H2 derived from YC244 clone)
RNNANGNT

SEQ ID NO: 26 - protein, 8 aa, Artificial Sequence
  Synthetic (CDR-H2 derived from YC321 clone)
RNKVNGYT

SEQ ID NO: 27 - protein, 6 aa, Artificial Sequence
  Synthetic (CDR-H3 derived from YC354 clone)
DNWLSY

SEQ ID NO: 28 - protein, 6 aa, Artificial Sequence
  Synthetic (CDR-H3 derived from YC374 clone)
DNWLTY

SEQ ID NO: 29 - protein, 17 aa, Artificial Sequence
  Synthetic (CDR-L1 derived from L1-1 clone)
KSSHSLLASGNQNNYLA

SEQ ID NO: 30 - protein, 17 aa, Artificial Sequence
  Synthetic (CDR-L1 derived from L1-3 clone)
KSSRSLLSSGNHKNYLA

SEQ ID NO: 31 - protein, 17 aa, Artificial Sequence
  Synthetic (CDR-L1 derived from L1-4 clone)
KSSKSLLASGNQNNYLA

SEQ ID NO: 32 - protein, 17 aa, Artificial Sequence
  Synthetic (CDR-L1 derived from L1-12 clone)
KSSRSLLASGNQNNYLA

SEQ ID NO: 33 - protein, 17 aa, Artificial Sequence
  Synthetic (CDR-L1 derived from L1-22 clone)
KSSHSLLASGNQNNYLA

SEQ ID NO: 34 - protein, 7 aa, Artificial Sequence
  Synthetic (CDR-L2 derived from L2-9 clone)
WASKRVS

SEQ ID NO: 35 - protein, 7 aa, Artificial Sequence
  Synthetic (CDR-L2 derived from L2-12 clone)
WGSTRVS

SEQ ID NO: 36 - protein, 7 aa, Artificial Sequence
  Synthetic (CDR-L2 derived from L2-16 clone)
WGSTRVP

SEQ ID NO: 37 - protein, 9 aa, Artificial Sequence
  Synthetic (CDR-L3 derived from L3-32 clone)
QQSYSKPFT

SEQ ID NO: 38 - DNA, 1416 bp, Artificial Sequence
  Synthetic (nucleotide sequence of heavy chain of chAbF46)
gaattcgccgccaccatggaatggagctgggtttttctcgtaacacttttaaatggtatccagtgtgaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatggacaccctgagagctgaggacagtgccacttattactgtgcaagagataactggtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 39 - DNA, 759 bp, Artificial Sequence
  Synthetic (nucleotide sequence of light chain of chAbF46)
gaattcactagtgattaattcgccgccaccatggattcacaggcccaggtcctcatgttgctgctgctatcggtatctggtacctgtggagacattttgatgacccagtctccatcctccctgactgtgtcagcaggagagaaggtcactatgagctgcaagtccagtcagagtcttttagctagtggcaaccaaaataactacttggcctggcaccagcagaaaccaggacgatctcctaaaatgctgataatttgggcatccactagggtatctggagtccctgatcgcttcataggcagtggatctgggacggatttcactctgaccatcaacagtgtgcaggctgaagatctggctgtttattactgtcagcagtcctacagcgctccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 40 - protein, 447 aa, Artificial Sequence
  Synthetic (amino acid sequence of H1-heavy)
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 447 aa, Artificial Sequence
  Synthetic (amino acid sequence of H3-heavy)
EVQLVESGGGLVQPGGSLRLSCAASGFTFTDYYMSWVRQAPGKGLEWLGFIRNKANGYTTEYSASVKGRFTISRDNSKNSLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 447 aa, Artificial Sequence
  Synthetic (amino acid sequence of H4-heavy)
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 220 aa, Artificial Sequence
  Synthetic (amino acid sequence of H1-light)
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 220 aa, Artificial Sequence
  Synthetic (amino acid sequence of H2-light)
DIVMTQTPLSLPVTPGEPASISCKSSQSLLASGNQNNYLAWHLQKPGQSPQMLIIWASTRVSGVPDRFSGSGSGTDFTLKISRVEAEDVGVYYCQQSYSAPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 220 aa, Artificial Sequence
  Synthetic (amino acid sequence of H3-light)
DIVMTQSPDSLAVSLGERATINCKSSQSLLASGNQNNYLAWYQQKPGQPPKLLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 219 aa, Artificial Sequence
  Synthetic (amino acid sequence of H4-light)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 47 - DNA, 1350 bp, Artificial Sequence
  Synthetic (nucleotide sequence of H1-heavy)
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgaaaaccgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 48 - DNA, 1350 bp, Artificial Sequence
  Synthetic (nucleotide sequence of H3-heavy)
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgcgtgctgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 49 - DNA, 1350 bp, Artificial Sequence
  Synthetic (nucleotide sequence of H4-heavy)
gaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 50 - DNA, 669 bp, Artificial Sequence
  Synthetic (nucleotide sequence of H1-light)
gacatcgtgatgacccagtctccagactccctggctgtgtctctgggcgagagggccaccatcaactgcaagtccagccagagtcttttagctagcggcaaccaaaataactacttagcttggcaccagcagaaaccaggacagcctcctaagatgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 51 - DNA, 669 bp, Artificial Sequence
  Synthetic (nucleotide sequence of H2-light)
gatattgtgatgacccagactccactctccctgcccgtcacccctggagagccggcctccatctcctgcaagtccagtcagagtcttttagctagtggcaaccaaaataactacttggcctggcacctgcagaagccagggcagtctccacagatgctgatcatttgggcatccactagggtatctggagtcccagacaggttcagtggcagtgggtcaggcactgatttcacactgaaaatcagcagggtggaggctgaggatgttggagtttattactgccagcagtcctacagcgctccgctcacgttcggacagggtaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 52 - DNA, 669 bp, Artificial Sequence
  Synthetic (nucleotide sequence of H3-light)
gacatcgtgatgacccagtctccagactccctggctgtgtctctgggcgagagggccaccatcaactgcaagtccagccagagtcttttagctagcggcaaccaaaataactacttagcttggtaccagcagaaaccaggacagcctcctaagctgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 53 - DNA, 669 bp, Artificial Sequence
  Synthetic (nucleotide sequence of H4-light)
gatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagcgctccgctcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 54 - protein, 23 aa, Artificial Sequence
  Synthetic (linker between VH and VL)
GLGGLGGGGSGGGGSGGSSGVGS

SEQ ID NO: 55 - DNA, 1088 bp, Artificial Sequence
  Synthetic (polynucleotide encoding scFv of huAbF46 antibody)
gctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaac

SEQ ID NO: 56 - DNA, 5597 bp, Artificial Sequence
  Synthetic (expression vector including polynucleotide encoding scFv of huAbF46 antibody)
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagctaattctacttcatacattttcaattaagatgcagttacttcgctgtttttcaatattttctgttattgctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaacccgctgatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccgtgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaatttcttgaagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctggctagt

SEQ ID NO: 57 - protein, 13 aa, Artificial Sequence
  Synthetic (U6-HC7 hinge)
EPKSCDCHCPPCP

SEQ ID NO: 58 - DNA, 435 bp, Artificial Sequence
  Synthetic (polynucleotide encoding CDR-L3 derived from L3-1 clone)
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacg

SEQ ID NO: 59 - DNA, 435 bp, Artificial Sequence
  Synthetic (polynucleotide encoding CDR-L3 derived from L3-2 clone)
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgggcagtcctacagccgtccgctcacgttcggacagggtaccaaggtggagatcaaacgtacg

SEQ ID NO: 60 - DNA, 435 bp, Artificial Sequence
  Synthetic (polynucleotide encoding CDR-L3 derived from L3-3 clone)
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgcacagtcctacagccatccgttctctttcggacagggtaccaaggtggagatcaaacgtacg

SEQ ID NO: 61 - DNA, 435 bp, Artificial Sequence
  Synthetic (polynucleotide encoding CDR-L3 derived from L3-5 clone)
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtttacgttcggacagggtaccaaggtggagatcaaacgtacg

SEQ ID NO: 62 - protein, 462 aa, Artificial Sequence
  Synthetic (polypeptide consisting of heavy chain of huAbF46-H4-A1, U6-HC7 hinge and constant region of human IgG1)
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1410 bp, Artificial Sequence
  Synthetic (polynucleotide encoding polypeptide consisting of heavy chain of huAbF46-H4-A1, U6-HC7 hinge and constant region of human IgG1)
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaaagctgcgattgccactgtcctccatgtccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 64 - protein, 461 aa, Artificial Sequence
  Synthetic (polypeptide consisting of heavy chain of huAbF46-H4-A1, human IgG2 hinge and constant region of human IgG1)
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1407 bp, Artificial Sequence
  Synthetic (polynucleotide encoding polypeptide consisting of heavy chain of huAbF46-H4-A1, human IgG2 hinge and constant region of human IgG1)
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagaggaagtgctgtgtggagtgccccccctgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 66 - protein, 460 aa, Artificial Sequence
  Synthetic (polypeptide consisting of heavy chain of huAbF46-H4-A1, human IgG2 hinge and constant region of human IgG2)
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - DNA, 1404 bp, Artificial Sequence
  Synthetic (polynucleotide encoding polypeptide consisting of heavy chain of huAbF46-H4-A1, human IgG2 hinge and constant region of human IgG2)
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 68 - protein, 240 aa, Artificial Sequence [sequence omitted]

SEQ ID NO: 69 - DNA, 758 bp, Artificial Sequence
  Synthetic (polynucleotide encoding polypeptide consisting of light chain of huAbF46-H4-A1(H36Y) and human kappa constant region)
aattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggtaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 70 - protein, 240 aa, Artificial Sequence
  Synthetic (polypeptide consisting of light chain of huAbF46-H4-A1 and human  kappa constant region)
MDSQAQVLMLLLLSVSGTCGDIQMTQSPSSLSASVGDRVTITCKSSQSLLASGNQNNHLAWYQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 19 aa, Artificial Sequence
  Synthetic (epitope in SEMA domain of c-Met)
FSPQIEEPSQCPDCVVSAL

SEQ ID NO: 72 - protein, 10 aa, Artificial Sequence
  Synthetic (epitope in SEMA domain of c-Met)
PQIEEPSQCP

SEQ ID NO: 73 - protein, 5 aa, Artificial Sequence
  Synthetic (epitope in SEMA domain of c-Met)
EEPSQ

SEQ ID NO: 74 - protein, 117 aa, Artificial Sequence
  Synthetic (heavy chain variable region of anti-c-Met antibody (AbF46 or huAbF46-H1))
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSS

SEQ ID NO: 75 - protein, 114 aa, Artificial Sequence
  Synthetic (light chain variable region of anti-c-Met antibody (AbF46 or  huAbF46-H1))
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKR

SEQ ID NO: 76 - DNA, 1416 bp, Artificial Sequence; identical to SEQ ID NO: 38
  Synthetic (nucleotide sequence of heavy chain of anti-c-Met antibody (AbF46 or huAbF46-H1))

SEQ ID NO: 77 - DNA, 759 bp, Artificial Sequence; identical to SEQ ID NO: 39
  Synthetic (nucleotide sequence of light chain of anti-c-Met antibody (AbF46 or huAbF46-H1))

SEQ ID NO: 78 - DNA, 4170 bp, Artificial Sequence
  Synthetic (polynucleotide encoding c-Met protein)
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 79 - protein, 444 aa, Artificial Sequence
  Synthetic (SEMA domain of c-Met)
LHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNG

SEQ ID NO: 80 - protein, 451 aa, Artificial Sequence
  Synthetic (PSI-IPT domain of c-Met)
YTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTG

SEQ ID NO: 81 - protein, 313 aa, Artificial Sequence
  Synthetic (TyrKc domain of c-Met)
VHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 82 - DNA, 1332 bp, Artificial Sequence
  Synthetic (polynucleotide encoding SEMA domain of c-Met)
ctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggc

SEQ ID NO: 83 - DNA, 1299 bp, Artificial Sequence
  Synthetic (polynucleotide encoding PSI-IPT domain of c-Met)
tacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacagga

SEQ ID NO: 84 - DNA, 939 bp, Artificial Sequence
  Synthetic (polynucleotide encoding TyrKc domain of c-Met)
gtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 85 - protein, 13 aa, Artificial Sequence
  Synthetic (heavy chain CDR3 of anti-c-Met antibody)
DNWFAYWGQGTLV

SEQ ID NO: 86 - protein, 10 aa, Artificial Sequence
  Synthetic (light chain CDR3 of anti-c-Met antibody)
LTFGAGTKLE

SEQ ID NO: 87 - protein, 117 aa, Artificial Sequence
  Synthetic (heavy chain variable region of monoclonal antibody AbF46)
EVKLVESGGGLVQPGGSLRLSCATSGFTFTDYYMSWVRQPPGKALEWLGFIRNKANGYTTEYSASVKGRFTISRDNSQSILYLQMDTLRAEDSATYYCARDNWFAYWGQGTLVTVSA

SEQ ID NO: 88 - protein, 114 aa, Artificial Sequence
  Synthetic (light chain variable region of anti-c-Met antibody)
DILMTQSPSSLTVSAGEKVTMSCKSSQSLLASGNQNNYLAWHQQKPGRSPKMLIIWASTRVSGVPDRFIGSGSGTDFTLTINSVQAEDLAVYYCQQSYSAPLTFGAGTKLELKR

SEQ ID NO: 89 - protein, 17 aa, Artificial Sequence
  Synthetic (light chain CDR3 of anti-c-Met antibody)
QQSYSAPLTFGAGTKLE

SEQ ID NO: 90 - protein, 117 aa, Artificial Sequence
  Synthetic (heavy chain variable region of AT-VH1)
EVKLVESGGGLVQPGGSLRLSCATSGFTFTDYYMSWVRQPPGKGLEWLGFIRNKANGYTTEYSASVKGRFTISRDNSKSTLYLQMNSLRAEDSATYYCARDNWFAYWGQGTLVTVSS

SEQ ID NO: 91 - protein, 117 aa, Artificial Sequence
  Synthetic (heavy chain variable region of AT-VH2)
EVKLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 92 - protein, 117 aa, Artificial Sequence
  Synthetic (heavy chain variable region of AT-VH3)
EVQLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 93 - protein, 117 aa, Artificial Sequence
  Synthetic (heavy chain variable region of AT-VH4)
EVQLVESGGGLVQPGGSLRLSCATSGFTFTDYYMSWVRQPPGKGLEWLGFIRNKANGYTTEYSASVKGRFTISRDNSKNTLYLQMNSLRAEDTATYYCARDNWFAYWGQGTLVTVSS

SEQ ID NO: 94 - protein, 117 aa, Artificial Sequence
  Synthetic (heavy chain variable region of AT-VH5)
EVQLVESGGGLVQPGGSLRLSCATSGFTFTDYYMSWVRQPPGKGLEWLGFIRNKANGYTTEYSASVKGRFTISRDNSKNTLYLQMNSLRAEDTAVYYCARDNWFAYWGQGTLVTVSS

SEQ ID NO: 95 - protein, 114 aa, Artificial Sequence
  Synthetic (light chain variable region of anti c-Met humanized antibody(huAbF46-H4))
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKR

SEQ ID NO: 96 - protein, 113 aa, Artificial Sequence
  Synthetic (light chain variable region of AT-Vk1)
DILMTQSPSSLTASVGDRVTMTCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 97 - protein, 113 aa, Artificial Sequence
  Synthetic (light chain variable region of AT-Vk2)
DILMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 98 - protein, 113 aa, Artificial Sequence
  Synthetic (light chain variable region of AT-Vk3)
DIQMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 99 - protein, 113 aa, Artificial Sequence
  Synthetic (light chain variable region of AT-Vk4)
DIQMTQSPSSLSASVGDRVTITCKSSQSLLASGNQNNYLAWHQQKPGKAPKMLIIWASTRVSGVPDRFSGSGSGTDFTLTISSLQAEDVAVYYCQQSYSAPLTFGQGTKLEIK

SEQ ID NO: 100 - protein, 13 aa, Artificial Sequence
  Synthetic (modified hinge region(U7-HC6))
EPSCDKHCCPPCP

SEQ ID NO: 101 - protein, 13 aa, Artificial Sequence
  Synthetic (modified hinge region(U6-HC7))
EPKSCDCHCPPCP

SEQ ID NO: 102 - protein, 12 aa, Artificial Sequence
  Synthetic (modified hinge region(U3-HC9))
ERKCCVECPPCP

SEQ ID NO: 103 - protein, 14 aa, Artificial Sequence
  Synthetic (modified hinge region(U6-HC8))
EPRDCGCKPCPPCP

SEQ ID NO: 104 - protein, 13 aa, Artificial Sequence
  Synthetic (modified hinge region(U8-HC5))
EKCDKTHTCPPCP

SEQ ID NO: 105 - protein, 15 aa, Artificial Sequence
  Synthetic (human hinge region)
EPKSCDKTHTCPPCP

SEQ ID NO: 106 - protein, 17 aa, Artificial Sequence
  Synthetic (CDR-L1 of antibody L3-11Y)
KSSQSLLAWGNQNNYLA

SEQ ID NO: 107 - protein, 114 aa, Artificial Sequence
  Synthetic (amino acid sequence of light chain variable region of antibody L3-11Y)
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKR

SEQ ID NO: 108 - protein, 220 aa, Artificial Sequence
  Synthetic (amino acid sequence of light chain of antibody L3-11Y)
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 1118 aa, Artificial Sequence
  Synthetic (human USP8 (NP_001122082))
MPAVASVPKELYLSSSLKDLNKKTEVKPEKISTKSYVHSALKIFKTAEECRLDRDEERAYVLYMKYVTVYNLIKKRPDFKQQQDYFHSILGPGNIKKAVEEAERLSESLKLRYEEAEVRKKLEEKDRQEEAQRLQQKRQETGREDGGTLAKGSLENVLDSKDKTQKSNGEKNEKCETKEKGAITAKELYTMMTDKNISLIIMDARRMQDYQDSCILHSLSVPEEAISPGVTASWIEAHLPDDSKDTWKKRGNVEYVVLLDWFSSAKDLQIGTTLRSLKDALFKWESKTVLRNEPLVLEGGYENWLLCYPQYTTNAKVTPPPRRQNEEVSISLDFTYPSLEESIPSKPAAQTPPASIEVDENIELISGQNERMGPLNISTPVEPVAASKSDVSPIIQPVPSIKNVPQIDRTKKPAVKLPEEHRIKSESTNHEQQSPQSGKVIPDRSTKPVVFSPTLMLTDEEKARIHAETALLMEKNKQEKELRERQQEEQKEKLRKEEQEQKAKKKQEAEENEITEKQQKAKEEMEKKESEQAKKEDKETSAKRGKEITGVKRQSKSEHETSDAKKSVEDRGKRCPTPEIQKKSTGDVPHTSVTGDSGSGKPFKIKGQPESGILRTGTFREDTDDTERNKAQREPLTRARSEEMGRIVPGLPSGWAKFLDPITGTFRYYHSPTNTVHMYPPEMAPSSAPPSTPPTHKAKPQIPAERDREPSKLKRSYSSPDITQAIQEEEKRKPTVTPTVNRENKPTCYPKAEISRLSASQIRNLNPVFGGSGPALTGLRNLGNTCYMNSILQCLCNAPHLADYFNRNCYQDDINRSNLLGHKGEVAEEFGIIMKALWTGQYRYISPKDFKITIGKINDQFAGYSQQDSQELLLFLMDGLHEDLNKADNRKRYKEENNDHLDDFKAAEHAWQKHKQLNESIIVALFQGQFKSTVQCLTCHKKSRTFEAFMYLSLPLASTSKCTLQDCLRLFSKEEKLTDNNRFYCSHCRARRDSLKKIEIWKLPPVLLVHLKRFSYDGRWKQKLQTSVDFPLENLDLSQYVIGPKNNLKKYNLFSVSNHYGGLDGGHYTAYCKNAARQRWFKFDDHEVSDISVSSVKSSAAYILFYTSLGPRVTDVAT

SEQ ID NO: 110 - DNA, 5704 bp, Artificial Sequence
  Synthetic (human USP8 coding gene, wherein nucleptide sequence from 339th to 3695th positions is coding region (CDS))
aatgcaaatcgggaaaagggggtgagctgggctggcttccgtcctggtagccaaggctaattctccctcgagttcttgggagatgggcatttggcgagaaggctggcgttagtgaagcgcgcccggcgtcacggtgagtgcgggtcttgggccctagcacctgttctctgggaagtcgtccgctgtgaacgatgaacgcctttccttccaccagctgctggttaccccggagacaagctctgtccgcggagaggagtgggacaactcctaaaggaaagaagcacttgtaaggaaatatagcatccattgtgaaagtggaaaagtaaagataattcatcatgcctgctgtggcttcagttcctaaagaactctacctcagttcttcactaaaagaccttaataagaagacagaagttaaaccagagaaaataagcactaagagttatgtgcacagtgccctgaagatctttaagacagcagaagaatgcagattagatcgtgatgaggaaagggcctatgtactatatatgaaatacgtgactgtttataatcttatcaaaaaaagacctgatttcaagcaacagcaggattatttccattcaatacttggacctggaaacatcaaaaaagctgtcgaagaagctgaaagactctctgaaagccttaaattaagatatgaagaagctgaagtccggaaaaaacttgaggaaaaagacaggcaggaggaagcacagcggctacaacaaaaaaggcaggaaacaggaagagaggatggtggcacattggctaaaggctctttggagaatgttttggattccaaagacaaaacccaaaagagcaatggtgaaaagaatgaaaaatgtgagaccaaagagaaaggagcaatcacagcaaaggaactatacacaatgatgacggataaaaacatcagcttgattataatggatgctcgaagaatgcaggattatcaggattcctgtattttacattctctcagtgttcctgaagaagccatcagtccaggagtcactgctagttggattgaagcacacctgccagatgattctaaagacacatggaagaagagggggaatgtggagtatgtggtacttcttgactggtttagttctgccaaagatttacagattggaacaactctccggagtctgaaagatgcacttttcaagtgggaaagtaaaactgtcctgcgcaatgagcctttggttttagagggaggctatgaaaactggctcctttgttatccccagtatacaacaaatgctaaggtcactccacccccacgacgccagaatgaagaggtgtctatctcattggattttacttatccctcattggaagaatcaattccttctaaacctgctgcccagacgccacctgcatctatagaagtagatgaaaatatagaattgataagtggtcaaaatgagagaatgggaccactgaatatatcaactccagttgaaccagttgctgcttctaaatctgatgtttcacccataattcagccagtgcctagtataaagaatgttccacagattgatcgtactaaaaaaccagcagtcaaattgcctgaagagcatagaataaaatctgaaagtacaaaccatgagcaacaatctcctcagagtggaaaagttattcctgatcgttccaccaagccagtagttttttctccaactctcatgttaacagatgaagaaaaggctcgtattcatgcagaaactgctcttctaatggaaaaaaacaaacaagaaaaagaacttcgggaaaggcagcaagaggaacagaaagagaaactgaggaaggaagaacaagaacaaaaagccaaaaagaaacaagaagctgaagaaaatgaaattacagagaagcaacaaaaagcaaaagaagaaatggagaagaaagaaagtgaacaggccaagaaagaagataaagaaacctcagcaaagaggggcaaagaaataacaggagtaaaaagacaaagtaaaagtgaacatgaaacttctgatgccaagaaatctgtagaagatagggggaaaaggtgtccaaccccagaaatacagaaaaagtcaacaggagatgtgccccatacatctgtgacaggggattcaggttcaggcaagccatttaagattaaaggacaaccagaaagtggaattctaaggacaggaacttttagagaggatacagacgataccgaaagaaataaagctcaacgagaacctttgacaagagcacgaagtgaagaaatggggaggatcgtaccaggactgccttcaggctgggccaagtttcttgacccaatcactggaacctttcgttattatcattcacccaccaacactgttcatatgtacccaccggaaatggctccttcatctgcacctccttccacccctccaactcataaagccaagccacagattcctgctgagcgggatagggaaccttccaaactgaagcgctcctactcctccccagatataacccaggctattcaagaggaagagaagaggaagccaacagtaactccaacagttaatcgggaaaacaagccaacatgttatcctaaagctgagatctcaaggctttctgcttctcagattcggaacctcaatcctgtttttggaggttctggaccagctcttactggacttcgtaacttaggaaatacttgttatatgaactcaatattgcagtgcctatgtaacgctccacatttggctgattatttcaaccgaaactgttatcaggatgatattaacaggtcaaatttgttggggcataaaggtgaagtggcagaagaatttggtataatcatgaaagccctgtggacaggacagtatagatatatcagtccaaaggactttaaaatcaccattgggaagatcaatgaccagtttgcaggatacagtcagcaagattcacaagaattgcttctgttcctaatggatggtctccatgaagatctaaataaagctgataatcggaagagatataaagaagaaaataatgatcatctcgatgactttaaagctgcagaacatgcctggcagaaacacaagcagctcaatgagtctattattgttgcactttttcagggtcaattcaaatctacagtacagtgcctcacatgtcacaaaaagtctaggacatttgaggccttcatgtatttgtctctaccactagcatccacaagtaaatgtacattacaggattgccttagattattttccaaagaagaaaaactcacagataacaacagattttactgcagtcattgcagagctcgacgggattctctaaaaaagatagaaatctggaagttaccacctgtgcttttagtgcatctgaaacgtttttcctacgatggcaggtggaaacaaaaattacagacatctgtggacttcccgttagaaaatcttgacttgtcacagtatgttattggtccaaagaacaatttgaagaaatataatttgttttctgtttcaaatcactacggtgggctggatggaggccactacacagcctattgtaaaaatgcagcaagacaacggtggtttaagtttgatgatcatgaagtttctgatatctccgtttcttctgtgaaatcttcagcagcttatatcctcttttatacttcattgggaccacgagtaactgatgtagccacataaggagacataggttataaactagttatcttttaaaaggctcagcaacacaactcttgaaatgcttatcaggataatggtagctatagctggccatttagaggaattctaggacagtgggagctgtgttactagcactatataattccggtcagtgctgacaaataacatttaacaagtattgcagtaatcatcacttacaggtaccatttatttcaaaacaacttttttagtctgctccaaagttaaaataattaactagctaagcattattattcgactggtctaaaaactattgttatcttttttttttccttttcactgttatggccttttcacatttctaaatcccatcttgatatactatgaatactctagaatgatgtaaagcagataggaatgtatgtgtacatatttattgcatacttgcacatcaaatcgatgtacatagtttaacacgtggtccttttgtgaaacctagaactcagaggattgctttttttctttcagcctattttgagttaacttcagtgctttcttagggaaatgacagggcaaagcaatttttctgttggctttgggctgtatttgtgcactaaatctttattctaaaaaaaaaaatggaaactttaatttttttaaaacgagaatttcatttacagctacattaaaatcttaatgagaaaaataatttataaccctgtgggtgttctgtctttaatattgtattatcaaatataggacagtaaaaccatagattttatatacacacgtgctatataataacacccagagtcattctttcaagactagtattctcacatattgagaatattcattctaaatattaaagtaaaaatgccgggagtcaggcatgattgcaaagtgaactgcattataaactacatctttacagagtgatgtattaagagggttaaaggagcttataatttatttaaccgagggactcagttgctatatatatagtcagtaaaacactccatataaaaataagattctaaaagtgcttcagaaagagaccaccattagcaggctctcagggagaagatgaaaggatggggttcaaattgtgaagctgacaacttttcatgttttacaattagtctaagagaccacttcttggctaaattattatatcaaatatattcaaatcatattcttaaactcatcgagccatttgaacaaaaattatttttgtttagcttcatgagtatctttggaaaataatttgttgaatatatatgattatgagatattttctgataaacactgaattttgaaacctgaactcactatataattgcagtgttttgaaggcctgcatccattagcattgcattatattcacactgccttttttagtgaaccaagacccatcttctggacgacagatttatcttaagatgaaaggttgtataacatgcccacaaggcataaaaatgttaatgatgcaagtaagttctaagagtttaatgaccaagcaaaactctaccaccagatgctgactgcttgttttgcagtgttcaggaaacaccattttcctggctcttaacgcttttgtattggtatggaaaagggctggcagctatagaacaggagatccatagcattttgaacagaagtatctggaatctcactgactcgtgtgttatcaaagctatatcaggcctgggtgactgaattcttgcagaaagcagtgtagtggccaccatccaaatcaccaaaatggttctatgggagaaaggaatgtcaaacttagtattcacatatgaacactaactactggaacagaaatgatagggccaagagatgctttttaaattgtcccttattctaaattaaaaggaagtgataattttgttgttaaatcatgcatatagcctgactgctatattgcttctcatttcattgtaactacttatatgttgtgcccattgactatcatctgtgaataaagaaagacaatatttagcaaaaaaaaaaaaaaaaaa

SEQ ID NO: 111 - protein, 1118 aa, Artificial Sequence
  Synthetic (human USP8 active site mutant (C786S))
MPAVASVPKELYLSSSLKDLNKKTEVKPEKISTKSYVHSALKIFKTAEECRLDRDEERAYVLYMKYVTVYNLIKKRPDFKQQQDYFHSILGPGNIKKAVEEAERLSESLKLRYEEAEVRKKLEEKDRQEEAQRLQQKRQETGREDGGTLAKGSLENVLDSKDKTQKSNGEKNEKCETKEKGAITAKELYTMMTDKNISLIIMDARRMQDYQDSCILHSLSVPEEAISPGVTASWIEAHLPDDSKDTWKKRGNVEYVVLLDWFSSAKDLQIGTTLRSLKDALFKWESKTVLRNEPLVLEGGYENWLLCYPQYTTNAKVTPPPRRQNEEVSISLDFTYPSLEESIPSKPAAQTPPASIEVDENIELISGQNERMGPLNISTPVEPVAASKSDVSPIIQPVPSIKNVPQIDRTKKPAVKLPEEHRIKSESTNHEQQSPQSGKVIPDRSTKPVVFSPTLMLTDEEKARIHAETALLMEKNKQEKELRERQQEEQKEKLRKEEQEQKAKKKQEAEENEITEKQQKAKEEMEKKESEQAKKEDKETSAKRGKEITGVKRQSKSEHETSDAKKSVEDRGKRCPTPEIQKKSTGDVPHTSVTGDSGSGKPFKIKGQPESGILRTGTFREDTDDTERNKAQREPLTRARSEEMGRIVPGLPSGWAKFLDPITGTFRYYHSPTNTVHMYPPEMAPSSAPPSTPPTHKAKPQIPAERDREPSKLKRSYSSPDITQAIQEEEKRKPTVTPTVNRENKPTCYPKAEISRLSASQIRNLNPVFGGSGPALTGLRNLGNTSYMNSILQCLCNAPHLADYFNRNCYQDDINRSNLLGHKGEVAEEFGIIMKALWTGQYRYISPKDFKITIGKINDQFAGYSQQDSQELLLFLMDGLHEDLNKADNRKRYKEENNDHLDDFKAAEHAWQKHKQLNESIIVALFQGQFKSTVQCLTCHKKSRTFEAFMYLSLPLASTSKCTLQDCLRLFSKEEKLTDNNRFYCSHCRARRDSLKKIEIWKLPPVLLVHLKRFSYDGRWKQKLQTSVDFPLENLDLSQYVIGPKNNLKKYNLFSVSNHYGGLDGGHYTAYCKNAARQRWFKFDDHEVSDISVSSVKSSAAYILFYTSLGPRVTDVAT

SEQ ID NO: 112 - DNA, 19 bp, Artificial Sequence
  Synthetic (shUSP8 mature antisense)
tatctcttccgattatcag